SEQUENCE LISTING (40 sequences)

SEQ ID NO: 1 - protein, 45 aa, Homo sapiens
QSVFKFWTHEGRKFWRQKLWPLWRWRPILCKTTKPRWLWRFQQQQ

SEQ ID NO: 2 - DNA, 180 bp, Homo sapiens
caatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaattaggaaacaaagcttt

SEQ ID NO: 3 - protein, 39 aa, Homo sapiens
GQMGVGRFSVWQPVGLGVGSGREPRGLGKGKDLGPCPGP

SEQ ID NO: 4 - DNA, 151 bp, Homo sapiens
gggcagatgggtgtcggcagatttagtgtttggcaaccagtggggctgggggtgggatctgggagggagccgaggggcctggggaagggaaaagatcttggaccctgccccggcccataggacactcaaaaacactttataaaaattgggg

SEQ ID NO: 5 - protein, 9 aa, Homo sapiens
ILYKNSSKE

SEQ ID NO: 6 - DNA, 416 bp, Homo sapiens
attttatacaaaaacagcagcaaagaataaagtaccaattgaaggtggcagttctgtctactgtgttctgcaaaaggctgggaaaagaagaggactcttttttcttttttccagttgaaataatttagactagttttctccctaattgctgctgggagcagtacttgttacacaatacaactgctatagttgtcaccggtgatcctctaccgggagctagatcatcttatatcccagggggaagatgacagacctctttgtaccaagcctgcaattttcaaaggtgtaaaaatattttaatagcccttttcatttgcctatgttggcctgagatgtttcccttctggtgaatttgcttctgtaaatttcagcttgcagaagtgctggcaggtatctcttcaacaaaacctggaaaa

SEQ ID NO: 7 - protein, 104 aa, Homo sapiens
NSEEKLEQSTIVKERGTVYFKEGKYKQALLQYKKIVSWLEYESSFSNEEAQKAQALRLASHLNLAMCHLKLQAFSAAIESCNKALELDSNNEKGLFRRGEAHLA

SEQ ID NO: 8 - DNA, 314 bp, Homo sapiens
aattcagaagagaagctggaacagagcaccatagtgaaagagcggggcactgtgtacttcaaggaaggtaaatacaagcaagctttactacagtataagaagatcgtgtcttggctggaatatgagtctagtttttccaatgaggaagcacagaaagcacaggcccttcgactggcctctcacctcaacctggccatgtgtcatctgaaactacaggccttctctgctgccattgaaagctgtaacaaggccctagaactggacagcaacaacgagaagggcctcttccgccggggagaggcccacctggccgt

SEQ ID NO: 9 - protein, 29 aa, Homo sapiens
LTVPHRALPHVLISFGCVKKWEKGKGESN

SEQ ID NO: 10 - DNA, 345 bp, Homo sapiens
ctgaccgtcccccacagggctctgcctcacgtcctcatctcatttggctgtgtaaagaaatgggaaaagggaaaaggagagagcaattgaggcagttgaccatattcagttttatttatttatttttaatttgtttttttctccaagtccaccagtctctgaaattagaacagtaggcggtatgagataatcaggcctaatcatgttgtgattctcttttcttagtggagtggaatgttctatccccacaagaaggattatatcttatagacttgtcttgttcagattctgtatttacccattttattgaaacatatactaagttccatgtatttttgttacaaa

SEQ ID NO: 11 - protein, 14 aa, Homo sapiens
GLTSKLSRRRVEEA

SEQ ID NO: 12 - DNA, 282 bp, Homo sapiens
ggattaacatcaaagttatccaggcgcagagttgaagaagcataagcaagacaaaaacagagagaccgcagaaggaggaagatactgtggtactgtcataaaaaacagtggagctctgtattagaaagcccctcagaactgggaaggccaggtaactctagttacacagaaactgtgactaaagtctatgaaactgattacaacagactgtaagaatcaaagtcaactgacatctatgctacatattattatatagtttgtactgagctattgaagtcccat

SEQ ID NO: 13 - protein, 24 aa, Homo sapiens
ARRLMGFSRVGLFSLMLVRVGKRG

SEQ ID NO: 14 - DNA, 289 bp, Homo sapiens
gccaggcgtttaatggggtttagtagggtggggttattttcgttaatgttagtaagggtggggaagcgaggttgacctgttagggtgagaagaattattcgagtgctataggcgcttgtcagggaggtagcgatgagagtaatagataggtgatatacatgatacattctcaagagttgcttgaccgaaagttacaaggaccccaacccctttgtcctctctacccacagatggccctgggaatcaattcctcaggaattgccctcaagaactctgcttcttgctttgc

SEQ ID NO: 15 - protein, 85 aa, Homo sapiens
EKIDRRSGKKLEDGPKFLKSGDAAIVDMVPGKPMCVESFSDYPPLGRFAVRDMRQTVAVGVIKAVDKKAAGAGKVTKSAQKAQKA

SEQ ID NO: 16 - DNA, 257 bp, Homo sapiens
gaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcagtggacaagaaggctgctggagctggcaaggtcaccaagtctgcccagaaagctcagaaggctaa

SEQ ID NO: 17 - protein, 31 aa, Homo sapiens
EVAAEEPNAAVPDEIPPLEGDEDASRMEEVD

SEQ ID NO: 18 - DNA, 360 bp, Homo sapiens
gaagtggcagcagaggaacccaatgctgcagttcctgatgagatcccccctctcgagggcgatgaggatgcgtctcgcatggaagaagtcgattaggttaggagttcatagttggaaaacttgtgcccttgtatagtgtccccatgggctcccactgcagcctcgagtgcccctgtcccacctggctccccctgctggtgtctagtgtttttttccctctcctgtccttgtgttgaaggcagtaaactaagggtgtcaagccccattccctctctactcttgacagcaggattggatgttgtgtattgtggtttattttattttcttcattttgttctgaaattaaagtatgcaaaataa

SEQ ID NO: 19 - protein, 85 aa, Homo sapiens
LNSIIDVYHKYSLIKGNFHAVYRDDLKKLLETECPQYIRKKGADVWFKELDINTDGAVNFQEFLILVIKMGVAAHKKSHEESHKE

SEQ ID NO: 20 - DNA, 692 bp, Homo sapiens
  misc_feature (685)..(685): n is a, c, g, or t
ttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtagctgagttactgggcccagaggctgggcccctggacatgtaaaaataattttaaaagcttacacagaaaatattattgcctgaagtttatgatctttaagttacaggtcaaaagagttttatgttgttgttgttgttgttgttgttttaaaacacacagtgaagaccgtctaagaagcaaggccactgttcctcctgtagggacaacgatcctgtctcagagacctgtccttctgctcctttagaaaacagaaccagcacagcgtccacaccgacagcagttactaggggcatagtcctccggtcgcagagggtggagttgaggcagagctcagtggtgtccacagaaagcattagtcttaggtactgtgttaaaacaatcaagaattcggatccccgagcatcacacctgactggatcgaaccccgnctccaag

SEQ ID NO: 21 - protein, 39 aa, Homo sapiens
RREGKSRWYFPALCMILDELYTQGFCSATPSSCVTEYQL

SEQ ID NO: 22 - DNA, 344 bp, Homo sapiens
cggagagagggaaaatcaaggtggtattttccagcactttgtatgattttggatgagttgtacacccaaggattctgttctgcaactccatcctcctgtgtcactgaatatcaactctgaaagagcaagcttgcggccgcactcgagtaactagttaaccccttggggcctctaaacgggtcttgaggggttaactagttactcgagtgcggccgcaagcttgctctttcagagttgatattcagtgacacaggaggatggagttgcagaacagaatccttgggtgtacaactcatccaaaatcatacaaagtgctggaaaataccacttgattttccctctct

SEQ ID NO: 23 - protein, 22 aa, Homo sapiens
QKCRLQRQYTGKGGVGFVYEGV

SEQ ID NO: 24 - DNA, 549 bp, Homo sapiens
cagaaatgtagactgcaaaggcagtatacaggaaaaggtggagtgggttttgtttatgagggtgtctgaaaactaaaattgagcgggatatcatggtatagttggacagtattggtccttcacactttggccatattgtataatggagcttttaccaaagatgtatgagaagtgtaagactataaaaaaatgaactattcaaagtaaaactcttaacaaacattttacttaaagcagatgcaaaagggtattctcatgtaggctcctgttggtgcagagggatttttttgatttcaggatacaactaaagtacgaagttctcagtttcactttagtagaaagagctctagaaatgaggctgataaacacatctaagaacactggttgctttctaaaatttccaaagctccaccataaatgtaatttttagtgtttcaaatgattgcattttaaagtatataaatatgggttatccaatatcaatgctatagtaacatcctgaaacaaaacaagcacaaaggtataaatgcctaaactggaggaagcttg

SEQ ID NO: 25 - protein, 120 aa, Homo sapiens
GRPPWPVSGVLGSRVCGPLYSTSPAGPGRAASLPRKGAQLELEEMLVPRKMSVSPLESWLTARCFLPRLDTGTAGTVAPPQSYQCPPSQIGEGAEQGDEGVADAPQIQCKNVLKIRRRKM

SEQ ID NO: 26 - DNA, 362 bp, Homo sapiens
ggccgcccgccttggcccgtctctggagtgctgggcagccgggtctgcgggcccctttacagcacatcgccggccggcccaggtagggcggcctctctccctcgcaagggggcccagctggagctggaggagatgctggtccccaggaagatgtccgtcagccccctggagagctggctcacggcccgctgcttcctgcccagactggataccgggaccgcagggactgtggctccaccgcaatcctaccagtgtccgcccagccagataggggaaggggccgagcagggggatgaaggcgtcgcggatgcgcctcaaattcagtgcaaaaacgtgctgaagatccgccggcggaagatgaa

SEQ ID NO: 27 - protein, 167 aa, Homo sapiens
SSCSEYNVRVASRYFKGPELLVDYQMYDYSLDMWSLGCMLASMIFRREPFFHGQDNYDQLVRIAKVLGTEELYGYLKKYHIDLDPHFNDILGQHSRKRWENFIHSENRHLVSPEALDLLDKLLRYDHQQRLTAKEAMEHPYFYPVVKEQSQPCADNAVLSSGLTAAR

SEQ ID NO: 28 - DNA, 523 bp, Homo sapiens
tcatcctgctcggagtacaatgttcgtgtagcctcaaggtacttcaagggaccagagctcctcgtggactatcagatgtatgattatagcttggacatgtggagtttgggctgtatgttagcaagcatgatctttcgaagggaaccattcttccatggacaggacaactatgaccagcttgttcgcattgccaaggttctgggtacagaagaactgtatgggtatctgaagaagtatcacatagacctagatccacacttcaacgatatcctgggacaacattcacggaaacgctgggaaaactttatccatagtgagaacagacaccttgtcagccctgaggccctagatcttctggacaaacttctgcgatacgaccatcaacagagactgactgccaaagaggccatggagcacccatacttctaccctgtggtgaaggagcagtcccagccttgtgcagacaatgctgtgctttccagtggtctcacggcagcacgatgaagactggaaagcgacgggt

SEQ ID NO: 29 - protein, 30 aa, Homo sapiens
GGRGGGGGGGGRGAGGGRGAGAGGGRPEAA

SEQ ID NO: 30 - DNA, 700 bp, Homo sapiens
ggaggtcgaggcggaggcggaggaggaggaggccgaggcgccggaggaggccgaggcgccggagcaggaggaggccggccggaggcggcatgagacgagcgtggcggccgcggctgctcggggccgcgctggttgcccattgacagcggcgtctgcagctcgcttcaagatggccgcttggctcgcattcattttctgctgaacgacttttaactttcattgtcttttccgcccgcttcgatcgcctcgcgccggctgctctttccgggattttttatcaagcagaaatgcgtcgaacaacgagaatcaagatcactgagctaaatccccacctgatgtgtgtgctttgtggagggtacttcattgatgccacaaccataatagaatgtctacattccttctgtaaaacgtgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggagaggttgcagatgaagataagagaattataactgatgatgagataataagcttgcggccgccctcgag

SEQ ID NO: 31 - protein, 69 aa, Homo sapiens
ARDLGEAALVAERKREEVTDVMASVHPLSLPRLLSPLAMLSSSFPVSSSGSYSTPIRKSLRRAAPPFRA

SEQ ID NO: 32 - DNA, 350 bp, Homo sapiens
gctagggacctcggagaagctgctctggtagctgagagaaagagggaggaggtgacagatgtgatggcctctgtgcatcctctgtcacttccgcgcctcctctctcccctcgccatgctctcctcttccttcccagtgagcagctccggctcctacagcactcccattcgcaagtccctgaggcgggcagcacctcctttcagggcataattgaggccaaccggaggtggctggaacgtgtcaagaatgaccccaggttacctctcttctcgtcaacacccaagccaaaagctactttgagcctcctgcatgctgggccttgatgagcacaatcagatgtgaaggtgtat

SEQ ID NO: 33 - protein, 19 aa, Homo sapiens
VSTFLSRVGRVSLLNFLPF

SEQ ID NO: 34 - DNA, 644 bp, Homo sapiens
gtttccacattcttgtcaagggttggtagggtcagtcttttaaatttcttgccattttagtgactgtgcattggtatttcattgtggtttatttgcatgatgactaatgctcaacaccaactaatcatgttgagtatttttaatgtgcttatttgccactcatatatcttctttgatgaagtgtctcttcaaatattttgcccatttaaaaactgtattgattcttattattgaattgcaataattctttctatccggatatatatcctttgccagatatgtgtattacaaatgttttctcctagccttccacctcagcctcccaagtagctgggaatgcaggtgtgcaccaccactccagggttttttgttgttgttgttgttgtttttctgtagagacagggtcttgccatgctgccgaggctgctctcaaactcctgggatcaagaaatcctcctgcctcggcctcccaaagtgctgacattacaagcatgagccactgtgcctggctaacttttcatcttttaaagtagtgtcttgcaaagaacaacattttaatgaagtccatttatcaactttttgattcattgtccatgctttttgcataataagaaatctttgcctgcctcaaaattgcaaagctt

SEQ ID NO: 35 - protein, 9 aa, Homo sapiens
FRGYLANNK

SEQ ID NO: 36 - DNA, 291 bp, Homo sapiens
tttaggggctatttagcaaataataaataaattgatttagaatagaagaaatcatgtgttggaaaagaggcttgaaacaagttcggtgttagagaagagaatattaagaaacaagtgggagataggacttctaaatgctacactaaggatttcggatttattctcatcctaaaggagagccagccaaggcttttctacaggagagaggtataatcaagaagcgtgaagctgagtcagtagggggatcagtgagaataggaagacatcagggttggggaagatgaaagctta

SEQ ID NO: 37 - protein, 89 aa, Homo sapiens
ESRPMSYDEKRQLSLDINKLPGEKLGRVVHIIQAREPSLRDSNPEEIEIDFETLKPSTLRELERYVLSCLRKKPRKPYSTYEMRFISWF

SEQ ID NO: 38 - DNA, 436 bp, Homo sapiens
gagagcaggcccatgagttacgatgagaagcggcagctgagcctggacatcaacaaattacctggggagaagctgggccgagttgtgcatataatccaagccagggagccctctttacgtgattcaaacccagaagagattgagattgattttgaaacactcaagccatccacacttagagagcttgagcgctatgtcctttcctgcctacgtaagaaaccccggaagccctacagtacgtatgaaatgaggttcatctcatggttctgaggacagttgaggaaagatggtggggtctgtttgcattcaggattgtcagctcccaggataatgggatgtgttggttggcagctgacgttcaagaagggaacttgggaaccttaggggcccataataagatgcttggggcaatcttaaggcttgcggccgcactcga

SEQ ID NO: 39 - protein, 116 aa, Homo sapiens
DPNQGGKDITEEIMSGARTASTPTPPQTGGGLEPQANGETPQVAVIVRPDDRSQGAIIADRPGLPGPEHSPSESQPSSPSPTPSPSPVLEPGSEPNLAVLSIPGDTMTTIQMSVEE

SEQ ID NO: 40 - DNA, 348 bp, Homo sapiens
gatccaaaccaaggaggaaaggatatcacagaggagatcatgtctggggcccgcactgcctccacacccacccctccccagacgggaggcggtctggagcctcaagctaatggggagacgccccaggttgctgtcattgtccggccagatgaccggtcacagggagcaatcattgctgaccggccagggctgcctggcccagagcatagcccttcagaatcccagccttcgtcgccttctccgaccccatcaccatccccagtcttggaaccggggtctgagcctaatctcgcagtcctctctattcctggggacactatgacaactatacaaatgtctgtagaagaa

(19) United States
(12) Patent Application Publication
(10) Pub. No.: US 2019/0137498 A1
(43) Pub. Date: May 9, 2019

(54) METHODS AND COMPOSITIONS FOR DETECTING CANCER

(71) Applicant: Exact Sciences Development Company, LLC, Madison, WI (US)

(72) Inventors: Jeanne Ohrnberger, Northville, MI (US); Sharat Singh, Rancho Santa Fe, CA (US); Jim Arthurs, Ann Arbor, MI (US); David Esposito, Portage, MI (US)

(21) Appl. No.: 16/182,854

(22) Filed: Nov. 7, 2018

Related U.S. Application Data
(60) Provisional application No. 62/582,893, filed on Nov. 7, 2017.

(51) Int. Cl. G01N 33/574; G16H 50/20; G06F 17/15

(52) U.S. Cl. CPC G01N 33/57434; G16H 50/30; G06F 17/15; G16H 50/20

(57) ABSTRACT

Disclosed herein are methods and composition of improving accuracy of cancer detection and enhancing therapeutic outcomes. The compositions, methods and systems described herein facilitate assessing risk assessment of cancer.

### CROSS-REFERENCE TO RELATED APPLICATIONS

The present application claims priority to U.S. Provisional Application Ser. No. 62/582,893, filed Nov. 7, 2017, which is incorporated herein by reference.

### BACKGROUND OF THE DISCLOSURE

According to the American Cancer Society, an estimated 180,890 new cases of prostate cancer were diagnosed in the United States in 2016, making this disease the most common solid tumor in men (Garisto and Klotz, Oncology 2017; 31(5):333-40, 345). The American Cancer Society estimates that about 161,360 American men will be diagnosed with prostate cancer and 26,730 will die in 2017.

Prostate cancer (PCA) is typically diagnosed with a digital rectal exam (DRE) and/or prostate specific antigen (PSA) screening. PSA, also known as gamma-seminoprotein or kallikrein-3 (KLK3), is secreted by the epithelial cells of the prostate gland and is used as a marker for prostate cancer. An elevated serum PSA level can indicate the presence of PCA. A healthy prostate will produce a stable amount—typically below 4 nanograms per milliliter (ng/mL), or a PSA reading of “4” or less—whereas cancer cells produce escalating amounts that correspond with the severity of the cancer. In general, the higher a man's PSA level, the more likely it is that he has prostate cancer. A continuous rise in a man's PSA level over time may also be a sign of prostate cancer. However, more recent studies have shown that some men with PSA levels below 4 ng/mL have prostate cancer and that many men with higher levels do not have prostate cancer (Thompson et al., New England Journal of Medicine 2004; 350(22): 2239-2246). In addition, various factors can cause a man's PSA level to fluctuate. For example, a man's PSA level often rises if he has prostatitis or a urinary tract infection. Prostate biopsies and prostate surgery also increase PSA level. Conversely, some drugs—including finasteride and dutasteride, which are used to treat benign prostatic hyperplasia (BPH)—lower a man's PSA level.

The advent of prostate specific antigen (PSA) screening has led to earlier detection of PCA and significantly reduced PCA-associated fatalities. However, a major limitation of the serum PSA test is a lack of prostate cancer sensitivity and specificity especially in the intermediate range of PSA detection (4-10 ng/ml). Elevated serum PSA levels are often detected in patients with non-malignant conditions such as benign prostatic hyperplasia (BPH) and prostatitis, and provide little information about the aggressiveness of the cancer detected. Moreover, due to the low specificity of the PSA assay and a high false positive rate, a large number of PCA biopsies are performed unnecessarily. Thus, development of additional serum and tissue biomarkers to supplement or replace PSA screening is needed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE DISCLOSURE

### Embodiment 1

A cancer profiling panel comprising a plurality of polypeptide probes, at least one of which comprises:
(a) a polypeptide probe comprising a full length or epitope fragment of a protein comprising amino acid sequence SEQ ID NO: 1 or encoded by SEQ ID NO: 2;
(b) a polypeptide probe comprising a full length or epitope fragment of a protein comprising amino acid sequence SEQ ID NO: 3 or encoded by SEQ ID NO: 4;
(c) a polypeptide probe comprising a full length or epitope fragment of a protein comprising amino acid sequence SEQ ID NO: 5 or encoded by SEQ ID NO: 6;
(d) a polypeptide probe comprising a full length or epitope fragment of a protein comprising amino acid sequence SEQ ID NO: 9 or encoded by SEQ ID NO: 10;
(e) a polypeptide probe comprising a full length or epitope fragment of a protein comprising amino acid sequence SEQ ID NO: 11 or encoded by SEQ ID NO: 12; or
(f) a polypeptide probe comprising a full length or epitope fragment of a protein comprising amino acid sequence SEQ ID NO: 13 or encoded by SEQ ID NO: 14.

### Embodiment 2

The cancer profiling panel of embodiment 1, further comprising:
(g) a polypeptide probe comprising the full length or epitope fragment of a protein comprising amino acid sequence SEQ ID NO: 7 or encoded by SEQ ID NO: 8;
(h) a polypeptide probe comprising the full length or epitope fragment of a protein comprising amino acid sequence SEQ ID NO: 15 or encoded by SEQ ID NO: 16;
(i) a polypeptide probe comprising the full length or epitope fragment of a protein comprising amino acid sequence SEQ ID NO: 17 or encoded by SEQ ID NO: 18;
(j) a polypeptide probe comprising the full length or epitope fragment of a protein comprising amino acid sequence SEQ ID NO: 19 or encoded by SEQ ID NO: 20;
(k) a polypeptide probe comprising the full length or epitope fragment of a protein comprising amino acid sequence SEQ ID NO: 21 or encoded by SEQ ID NO: 22;
(l) a polypeptide probe comprising the full length or epitope fragment of a protein comprising amino acid sequence SEQ ID NO: 23 or encoded by SEQ ID NO: 24;
(m) a polypeptide probe comprising the full length or epitope fragment of a protein comprising amino acid sequence SEQ ID NO: 25 or encoded by SEQ ID NO: 26;
(n) a polypeptide probe comprising the full length or epitope fragment of a protein comprising amino acid sequence SEQ ID NO: 27 or encoded by SEQ ID NO: 28;
(o) a polypeptide probe comprising the full length or epitope fragment of a protein comprising amino acid sequence SEQ ID NO: 29 or encoded by SEQ ID NO: 30;
(p) a polypeptide probe comprising the full length or epitope fragment of a protein comprising amino acid sequence SEQ ID NO: 31 or encoded by SEQ ID NO: 32;
(q) a polypeptide probe comprising the full length or epitope fragment of a protein comprising amino acid sequence SEQ ID NO: 33 or encoded by SEQ ID NO: 34;
(r) a polypeptide probe comprising the full length or epitope fragment of a protein comprising amino acid sequence SEQ ID NO: 35 or encoded by SEQ ID NO: 36;
(s) a polypeptide probe comprising the full length or epitope fragment of a protein comprising amino acid sequence SEQ ID NO: 37 or encoded by SEQ ID NO: 38; or
(t) a polypeptide probe comprising the full length or epitope fragment of a protein comprising amino acid sequence SEQ ID NO: 39 or encoded by SEQ ID NO: 40.

### Embodiment 3

The cancer profiling panel of embodiment 1 or 2, comprising two or more polypeptide probes selected from the group consisting of: (a), (b), and (c).

### Embodiment 4

The cancer profiling panel of embodiment 1 or 2, comprising polypeptide probes (a), (b), and (c).

### Embodiment 5

The cancer profiling panel of any one of embodiments 1-4, comprising two or more polypeptide probes selected from the group consisting of: (a), (b), (c), (d), (k), (l), (m), (n), (o), (p), (q), (r), (s), and (t).

### Embodiment 6

The cancer profiling panel of any one of embodiments 1-4, comprising three or more polypeptide probes selected from the group consisting of: (a), (b), (c), (d), (k), (l), (m), (n), (o), (p), (q), (r), (s), and (t).

### Embodiment 7

The cancer profiling panel of any one of embodiments 1-4, comprising four or more polypeptide probes selected from the group consisting of: (a), (b), (c), (d), (k), (l), (m), (n), (o), (p), (q), (r), (s), and (t).

### Embodiment 8

The cancer profiling panel of any one of embodiments 1-4, comprising five or more polypeptide probes selected from the group consisting of: (a), (b), (c), (d), (k), (l), (m), (n), (o), (p), (q), (r), (s), and (t).

### Embodiment 9

The cancer profiling panel of any one of embodiments 1-4, comprising six or more polypeptide probes selected from the group consisting of: (a), (b), (c), (d), (k), (l), (m), (n), (o), (p), (q), (r), (s), and (t).

### Embodiment 10

The cancer profiling panel of any one of embodiments 1-4, comprising seven or more polypeptide probes selected from the group consisting of: (a), (b), (c), (d), (k), (l), (m), (n), (o), (p), (q), (r), (s), and (t).

### Embodiment 11

The cancer profiling panel of any one of embodiments 1-4, comprising eight or more polypeptide probes selected from the group consisting of: (a), (b), (c), (d), (k), (l), (m), (n), (o), (p), (q), (r), (s), and (t).

### Embodiment 12

The cancer profiling panel of any one of embodiments 1-4, comprising nine or more polypeptide probes selected from the group consisting of: (a), (b), (c), (d), (k), (l), (m), (n), (o), (p), (q), (r), (s), and (t).

### Embodiment 13

The cancer profiling panel of any one of embodiments 1-4, comprising ten or more polypeptide probes selected from the group consisting of: (a), (b), (c), (d), (k), (l), (m), (n), (o), (p), (q), (r), (s), and (t).

### Embodiment 14

The cancer profiling panel of any one of embodiments 1-4, comprising polypeptide probes: (a), (b), (c), (k), (l), (m), (n), (o), (q), (r), (s), and (t).

### Embodiment 15

The cancer profiling panel of any one of embodiments 1-4, comprising polypeptide probes: (a), (b), (c), (d), (k), (l), (m), (n), (o), (p), (q), (r), (s), and (t).

### Embodiment 16

The cancer profiling panel of any one of embodiments 1-15, wherein the polypeptide probes are, individually, displayed on a phage.

### Embodiment 17

The cancer profiling panel of embodiment 16, wherein the phage is a T7 phage.

### Embodiment 18

The cancer profiling panel of any one of embodiments 1-17, wherein the polypeptide probes are configured to be specifically bound by an antibody.

### Embodiment 19

The cancer profiling panel of embodiment 18, wherein the antibody is a human antibody.

### Embodiment 20

The cancer profiling panel of embodiment 18 or 19, wherein the antibody is an autoantibody.

### Embodiment 21

The cancer profiling panel of any one of embodiments 1-20, wherein the polypeptide probes are arranged in an addressable array.

### Embodiment 22

The cancer profiling panel of embodiment 21, wherein the addressable array is a microarray.

### Embodiment 23

The cancer profiling panel of any one of embodiments 1-20, wherein the polypeptide probes are attached to a bead.

### Embodiment 24

The cancer profiling panel of any one of embodiments 1-20, wherein the polypeptide probes are attached to a man-made substrate.

### Embodiment 25

The cancer profiling panel of embodiment 24, wherein the man-made substrate is a bead or an array.

### Embodiment 26

The cancer profiling panel of any one of embodiments 1-25, wherein the epitope fragment is at least 2 amino acids in length.

### Embodiment 27

The cancer profiling panel of any one of embodiments 1-25, wherein the epitope fragment is at least 3 amino acids in length.

### Embodiment 28

The cancer profiling panel of any one of embodiments 1-25, wherein the epitope fragment is at least 4 amino acids in length.

### Embodiment 29

The cancer profiling panel of any one of embodiments 1-25, wherein the epitope fragment is at least 5 amino acids in length.

### Embodiment 30

The cancer profiling panel of any one of embodiments 1-25, wherein the epitope fragment is at least 6 amino acids in length.

### Embodiment 31

A method comprising contacting a sample from a subject with a cancer profiling panel comprising a plurality of polypeptide probes, at least one of which comprises:
(a) a polypeptide probe comprising a full length or epitope fragment of a protein comprising amino acid sequence SEQ ID NO: 1 or encoded by SEQ ID NO: 2;
(b) a polypeptide probe comprising a full length or epitope fragment of a protein comprising amino acid sequence SEQ ID NO: 3 or encoded by SEQ ID NO: 4;
(c) a polypeptide probe comprising a full length or epitope fragment of a protein comprising amino acid sequence SEQ ID NO: 5 or encoded by SEQ ID NO: 6;
(d) a polypeptide probe comprising a full length or epitope fragment of a protein comprising amino acid sequence SEQ ID NO: 9 or encoded by SEQ ID NO: 10;
(e) a polypeptide probe comprising a full length or epitope fragment of a protein comprising amino acid sequence SEQ ID NO: 11 or encoded by SEQ ID NO: 12; or
(f) a polypeptide probe comprising a full length or epitope fragment of a protein comprising amino acid sequence SEQ ID NO: 13 or encoded by SEQ ID NO: 14; and measuring a level of antibodies from the sample bound to the plurality of polypeptide probes.

### Embodiment 32

The method of embodiment 27, wherein the cancer profiling panel further comprises:
(g) a polypeptide probe comprising the full length or epitope fragment of a protein comprising amino acid sequence SEQ ID NO: 7 or encoded by SEQ ID NO: 8;
(h) a polypeptide probe comprising the full length or epitope fragment of a protein comprising amino acid sequence SEQ ID NO: 15 or encoded by SEQ ID NO: 16;
(i) a polypeptide probe comprising the full length or epitope fragment of a protein comprising amino acid sequence SEQ ID NO: 17 or encoded by SEQ ID NO: 18;
(j) a polypeptide probe comprising the full length or epitope fragment of a protein comprising amino acid sequence SEQ ID NO: 19 or encoded by SEQ ID NO: 20;
(k) a polypeptide probe comprising the full length or epitope fragment of a protein comprising amino acid sequence SEQ ID NO: 21 or encoded by SEQ ID NO: 22;
(l) a polypeptide probe comprising the full length or epitope fragment of a protein comprising amino acid sequence SEQ ID NO: 23 or encoded by SEQ ID NO: 24;
(m) a polypeptide probe comprising the full length or epitope fragment of a protein comprising amino acid sequence SEQ ID NO: 25 or encoded by SEQ ID NO: 26;
(n) a polypeptide probe comprising the full length or epitope fragment of a protein comprising amino acid sequence SEQ ID NO: 27 or encoded by SEQ ID NO: 28;
(o) a polypeptide probe comprising the full length or epitope fragment of a protein comprising amino acid sequence SEQ ID NO: 29 or encoded by SEQ ID NO: 30;
(p) a polypeptide probe comprising the full length or epitope fragment of a protein comprising amino acid sequence SEQ ID NO: 31 or encoded by SEQ ID NO: 32;
(q) a polypeptide probe comprising the full length or epitope fragment of a protein comprising amino acid sequence SEQ ID NO: 33 or encoded by SEQ ID NO: 34;
(r) a polypeptide probe comprising the full length or epitope fragment of a protein comprising amino acid sequence SEQ ID NO: 35 or encoded by SEQ ID NO: 36;
(s) a polypeptide probe comprising the full length or epitope fragment of a protein comprising amino acid sequence SEQ ID NO: 37 or encoded by SEQ ID NO: 38; or
(t) a polypeptide probe comprising the full length or epitope fragment of a protein comprising amino acid sequence SEQ ID NO: 39 or encoded by SEQ ID NO: 40.

### Embodiment 33

The method of embodiment 31 or 32, wherein the cancer profiling panel comprises two or more polypeptide probes selected from the group consisting of: (a), (b), and (c).

### Embodiment 34

The method of embodiment 31 or 32, wherein the cancer profiling panel comprises polypeptide probes (a), (b), and (c).

### Embodiment 35

The method of any one of embodiments 31-34, wherein the cancer profiling panel comprises two or more polypeptide probes selected from the group consisting of: (a), (b), (c), (d), (k), (l), (m), (n), (o), (p), (q), (r), (s), and (t).

### Embodiment 36

The method of any one of embodiments 31-34, wherein the cancer profiling panel comprises three or more polypeptide probes selected from the group consisting of: (a), (b), (c), (d), (k), (l), (m), (n), (o), (p), (q), (r), (s), and (t).

### Embodiment 37

The method of any one of embodiments 31-34, wherein the cancer profiling panel comprises four or more polypeptide probes selected from the group consisting of: (a), (b), (c), (d), (k), (l), (m), (n), (o), (p), (q), (r), (s), and (t).

### Embodiment 38

The method of any one of embodiments 31-34, wherein the cancer profiling panel comprises five or more polypeptide probes selected from the group consisting of: (a), (b), (c), (d), (k), (l), (m), (n), (o), (p), (q), (r), (s), and (t).

### Embodiment 39

The method of any one of embodiments 31-34, wherein the cancer profiling panel comprises six or more polypeptide probes selected from the group consisting of: (a), (b), (c), (d), (k), (l), (m), (n), (o), (p), (q), (r), (s), and (t).

### Embodiment 40

The method of any one of embodiments 31-34, wherein the cancer profiling panel comprises seven or more polypeptide probes selected from the group consisting of: (a), (b), (c), (d), (k), (l), (m), (n), (o), (p), (q), (r), (s), and (t).

### Embodiment 41

The method of any one of embodiments 31-34, wherein the cancer profiling panel comprises eight or more polypeptide probes selected from the group consisting of: (a), (b), (c), (d), (k), (l), (m), (n), (o), (p), (q), (r), (s), and (t).

### Embodiment 42

The method of any one of embodiments 31-34, wherein the cancer profiling panel comprises nine or more polypeptide probes selected from the group consisting of: (a), (b), (c), (d), (k), (l), (m), (n), (o), (p), (q), (r), (s), and (t).

### Embodiment 43

The method of any one of embodiments 31-34, wherein the cancer profiling panel comprises ten or more polypeptide probes selected from the group consisting of: (a), (b), (c), (d), (k), (l), (m), (n), (o), (p), (q), (r), (s), and (t).

### Embodiment 44

The method of any one of embodiments 31-34, wherein the cancer profiling panel comprises polypeptide probes: (a), (b), (c), (k), (l), (m), (n), (o), (q), (r), (s), and (t).

### Embodiment 45

The method of any one of embodiments 31-34, wherein the cancer profiling panel comprises polypeptide probes: (a), (b), (c), (d), (k), (l), (m), (n), (o), (p), (q), (r), (s), and (t).

### Embodiment 46

The method of any one of embodiments 31-45, wherein the polypeptide probes are, individually, displayed on a phage.

### Embodiment 47

The method of embodiment 46, wherein the phage is a T7 phage.

### Embodiment 48

The method of any one of embodiments 31-47, wherein the polypeptide probes are configured to be specifically bound by an antibody.

### Embodiment 49

The method of embodiment 48, wherein the antibody is a human antibody.

### Embodiment 50

The method of embodiment 48 or 49, wherein the antibody is an autoantibody.

### Embodiment 51

The method of any one of embodiments 31-50, wherein the polypeptide probes are arranged in an addressable array.

### Embodiment 52

The method of embodiment 51, wherein the addressable array is a microarray.

### Embodiment 53

The method of any one of embodiments 31-50, wherein the polypeptide probes are attached to a bead.

### Embodiment 54

The method of any one of embodiments 31-50, wherein the polypeptide probes are attached to a man-made substrate.

### Embodiment 55

The method of embodiment 54, wherein the man-made substrate is a bead or an array.

### Embodiment 56

The method of any one of embodiments 31-55, wherein the epitope fragment is at least 3 amino acids in length.

### Embodiment 57

The method of any one of embodiments 31-55, wherein the epitope fragment is at least 4 amino acids in length.

### Embodiment 58

The method of any one of embodiments 31-55, wherein the epitope fragment is at least 5 amino acids in length.

### Embodiment 59

The method of any one of embodiments 31-55, wherein the epitope fragment is at least 6 amino acids in length.

### Embodiment 60

The method of any one of embodiments 31-55, wherein the epitope fragment is at least 7 amino acids in length.

### Embodiment 61

The method of any one of embodiments 31-60, wherein the subject is suspected of having cancer.

### Embodiment 62

The method of any one of embodiments 31-60, wherein the subject is suspected of having prostate cancer.

### Embodiment 63

The method of any one of embodiments 31-62, wherein the subject has a PSA level of from about 2 to about 10 ng/mL.

### Embodiment 64

The method of any one of embodiments 31-62, wherein the subject has a PSA level of less than about 4 ng/mL.

### Embodiment 65

The method of any one of embodiments 31-64, wherein the level of antibodies from the sample bound to the plurality of polypeptide probes discriminates between subjects having a Gleason score of 6 or less and subjects having a Gleason score 7 or higher with a sensitivity of at least 60%, 70%, 80%, 85%, 90%, 91%, 92%, 93%, 94%, or 95%.

### Embodiment 66

The method of any one of embodiments 31-64, wherein the level of antibodies from the sample bound to the plurality of polypeptide probes discriminates between subjects having a Gleason score of 6 or less and subjects having a Gleason score 7 or higher with a sensitivity of at least 90%.

### Embodiment 67

The method of any one of embodiments 31-66, wherein the level of antibodies from the sample bound to the plurality of polypeptide probes discriminates between subjects having a Gleason score of 6 or less and subjects having a Gleason score 7 or higher with a specificity of at least 10%, 15%, 20%, 25%, 26%, 27%, 28%, 29%, 30%, 35%, 40%, 50%, 60%, 70%, 75%, 80%, 85%, 90%, or 95%.

### Embodiment 68

The method of any one of embodiments 31-66, wherein the level of antibodies from the sample bound to the plurality of polypeptide probes discriminates between subjects having a Gleason score of 6 or less and subjects having a Gleason score 7 or higher with a specificity of at least 20%.

### Embodiment 69

The method of any one of embodiments 31-68, wherein the level of antibodies from the sample bound to the plurality of polypeptide probes discriminates between subjects having a Gleason score of 6 or less and subjects having a Gleason score 7 or higher with a negative prediction value of at least 30%, 40%, 50%, 60%, 70%, 80%, 85%, 90%, 91%, 92%, 93%, 94%, or 95%.

### Embodiment 70

The method of any one of embodiments 31-68, wherein the level of antibodies from the sample bound to the plurality of polypeptide probes discriminates between subjects having a Gleason score of 6 or less and subjects having a Gleason score 7 or higher with a negative prediction value of at least 90%.

### Embodiment 71

The method of any one of embodiments 31-70, wherein the level of antibodies from the sample bound to the plurality of polypeptide probes discriminates between subjects having a Gleason score of 6 or less and subjects having a Gleason score 7 or higher with a positive prediction value of at least 10%, 15%, 20%, 25%, 26%, 27%, 28%, 29%, 30%, 35%, 40%, 50%, 60%, 70%, 80%, 85%, 90%, 91%, 92%, 93%, 94%, or 95%.

### Embodiment 72

The method of any one of embodiments 31-70, wherein the level of antibodies from the sample bound to the plurality of polypeptide probes discriminates between subjects having a Gleason score of 6 or less and subjects having a Gleason score 7 or higher with a positive prediction value of at least 40%.

### Embodiment 73

The method of any one of embodiments 31-72, wherein a binary classifier based on the level of antibodies from the sample bound to the plurality of polypeptide probes discriminates between subjects having a Gleason score of 6 or less and subjects having a Gleason score 7 or higher with an Area Under the Curve (AUC) of at least 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, or 0.9.

### Embodiment 74

The method of any one of embodiments 31-72, wherein a binary classifier based on the level of antibodies from the sample bound to the plurality of polypeptide probes discriminates between subjects having a Gleason score of 6 or less and subjects having a Gleason score 7 or higher with an Area Under the Curve (AUC) of at least 0.6.

### Embodiment 75

The method of any one of embodiments 31-72, wherein a binary classifier based on the level of antibodies from the sample bound to the plurality of polypeptide probes discriminates between subjects having a Gleason score of 6 or less and subjects having a Gleason score 7 or higher with an Area Under the Curve (AUC) of at least 0.8.

### Embodiment 76

The method of any one of embodiments 73-75, wherein the binary classifier is further based on the subjects age, PSA level, biopsy status, or both.

### Embodiment 77

The method of any one of embodiments 31-64, wherein the level of antibodies from the sample bound to the plurality of polypeptide probes discriminates between subjects having prostate cancer and not having prostate cancer with a sensitivity of at least 60%, 70%, 80%, 85%, 90%, 91%, 92%, 93%, 94%, or 95%.

### Embodiment 78

The method of any one of embodiments 31-64, wherein the level of antibodies from the sample bound to the plurality of polypeptide probes discriminates between subjects having prostate cancer and not having prostate cancer with a sensitivity of at least 90%.

### Embodiment 79

The method of any one of embodiments 31-66, wherein the level of antibodies from the sample bound to the plurality of polypeptide probes discriminates between subjects having prostate cancer and not having prostate cancer with a specificity of at least 10%, 15%, 20%, 25%, 26%, 27%, 28%, 29%, 30%, 35%, 40%, 50%, 60%, 70%, 75%, 80%, 85%, 90%, or 95%.

### Embodiment 80

The method of any one of embodiments 31-66, wherein the level of antibodies from the sample bound to the plurality of polypeptide probes discriminates between subjects having prostate cancer and not having prostate cancer with a specificity of at least 20%.

### Embodiment 81

The method of any one of embodiments 31-68, wherein the level of antibodies from the sample bound to the plurality of polypeptide probes discriminates between subjects having prostate cancer and not having prostate cancer with a negative prediction value of at least 30%, 40%, 50%, 60%, 70%, 80%, 85%, 90%, 91%, 92%, 93%, 94%, or 95%.

### Embodiment 82

The method of any one of embodiments 31-68, wherein the level of antibodies from the sample bound to the plurality of polypeptide probes discriminates between subjects having prostate cancer and not having prostate cancer with a negative prediction value of at least 90%.

### Embodiment 83

The method of any one of embodiments 31-70, wherein the level of antibodies from the sample bound to the plurality of polypeptide probes discriminates between subjects having prostate cancer and not having prostate cancer with a positive prediction value of at least 10%, 15%, 20%, 25%, 26%, 27%, 28%, 29%, 30%, 35%, 40%, 50%, 60%, 70%, 80%, 85%, 90%, 91%, 92%, 93%, 94%, or 95%.

### Embodiment 84

The method of any one of embodiments 31-70, wherein the level of antibodies from the sample bound to the plurality of polypeptide probes discriminates between subjects having prostate cancer and not having prostate cancer with a positive prediction value of at least 40%.

### Embodiment 85

The method of any one of embodiments 31-72, wherein a binary classifier based on the level of antibodies from the sample bound to the plurality of polypeptide probes discriminates between subjects having prostate cancer and not having prostate cancer with an Area Under the Curve (AUC) of at least 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, or 0.9.

### Embodiment 86

The method of any one of embodiments 31-72, wherein a binary classifier based on the level of antibodies from the sample bound to the plurality of polypeptide probes discriminates between subjects having prostate cancer and not having prostate cancer with an Area Under the Curve (AUC) of at least 0.6.

### Embodiment 87

The method of any one of embodiments 31-72, wherein a binary classifier based on the level of antibodies from the sample bound to the plurality of polypeptide probes discriminates between subjects having prostate cancer and not having prostate cancer with an Area Under the Curve (AUC) of at least 0.8.

### Embodiment 88

The method of any one of embodiments 85-87, wherein the binary classifier is further based on the subjects age, PSA level, biopsy status, or both.

### Embodiment 89

The method of any one of embodiments 31-88, wherein the sample is a blood or serum sample.

### BRIEF DESCRIPTION OF THE DRAWINGS

The features of the present disclosure are set forth with particularity in the appended claims. A better understanding of the features and advantages of the present will be obtained by reference to the following detailed description that sets forth illustrative embodiments, in which the principles of the disclosure are utilized, and the accompanying drawings of which:

FIG. 1 is an exemplary schematic overview of utilizing a serum-based antibody test as described herein in a primary care setting.

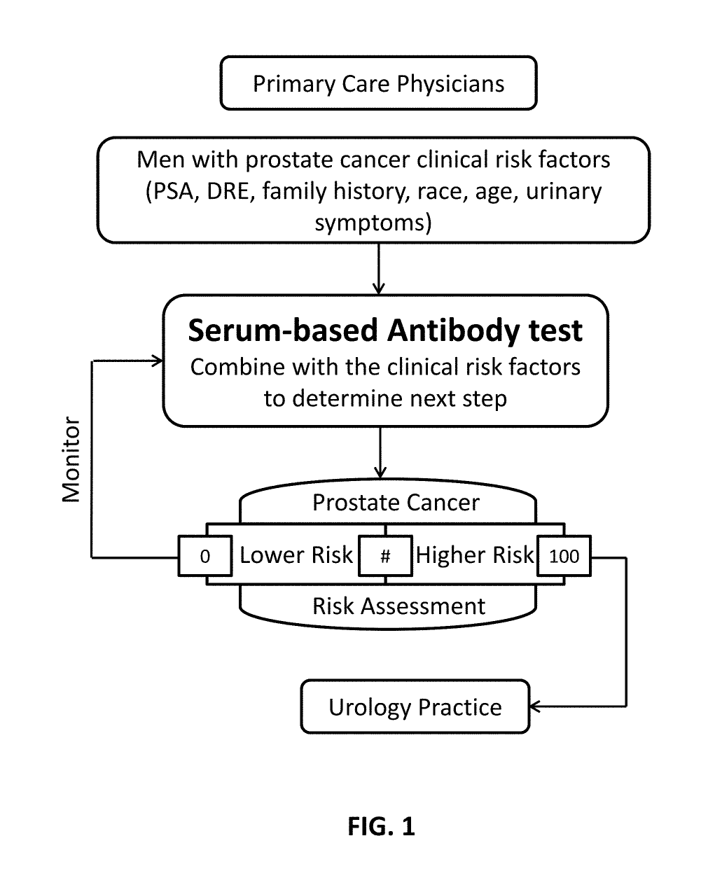

FIG. 2 is a schematic overview of utilizing an exemplary serum-based antibody test in a urology setting. A serum-based antibody test as described herein can be utilized after initial risk assessment for prostate cancer. The cancer active monitoring platform (active surveillance) can be utilized to help stratify risk in men with non-aggressive prostate cancer who are not receiving treatment.

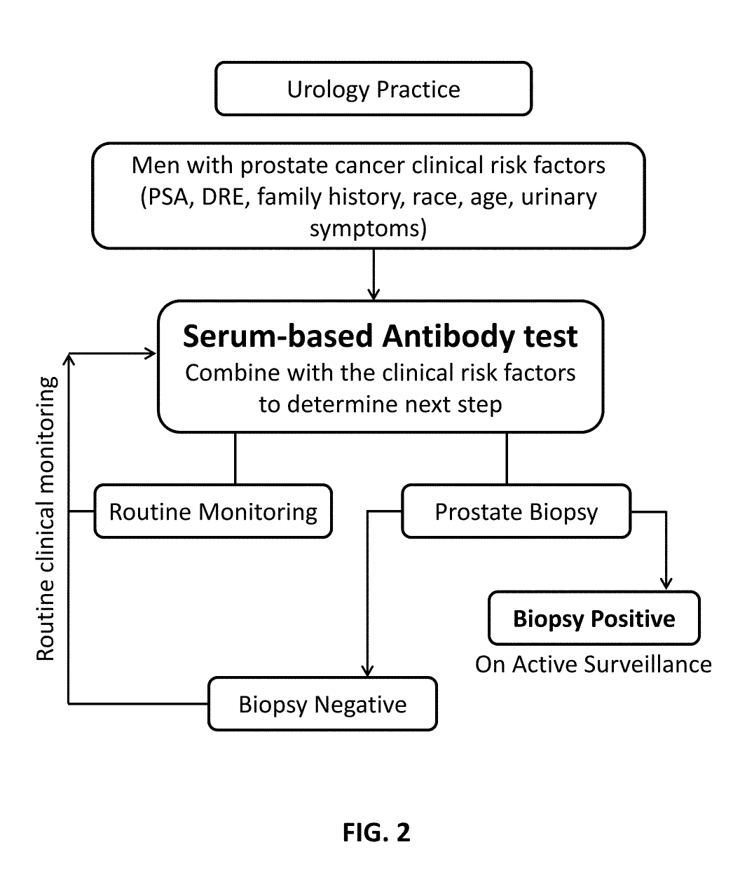

FIG. 3 is a ROC curve for a fitted model for prostate cancer detection from a training set when applied to a validation set and compared to the individual ROCs for 8 biomarkers.

FIG. 4 is a waterfall plot of the autoantibody assay score across the validation cohort for a prostate cancer prognosis panel.

FIG. 5 is a ROC curve for fitted model from the training set when applied to the validation set and compared to the individual ROCs for biomarkers selected for a prostate cancer monitoring panel.

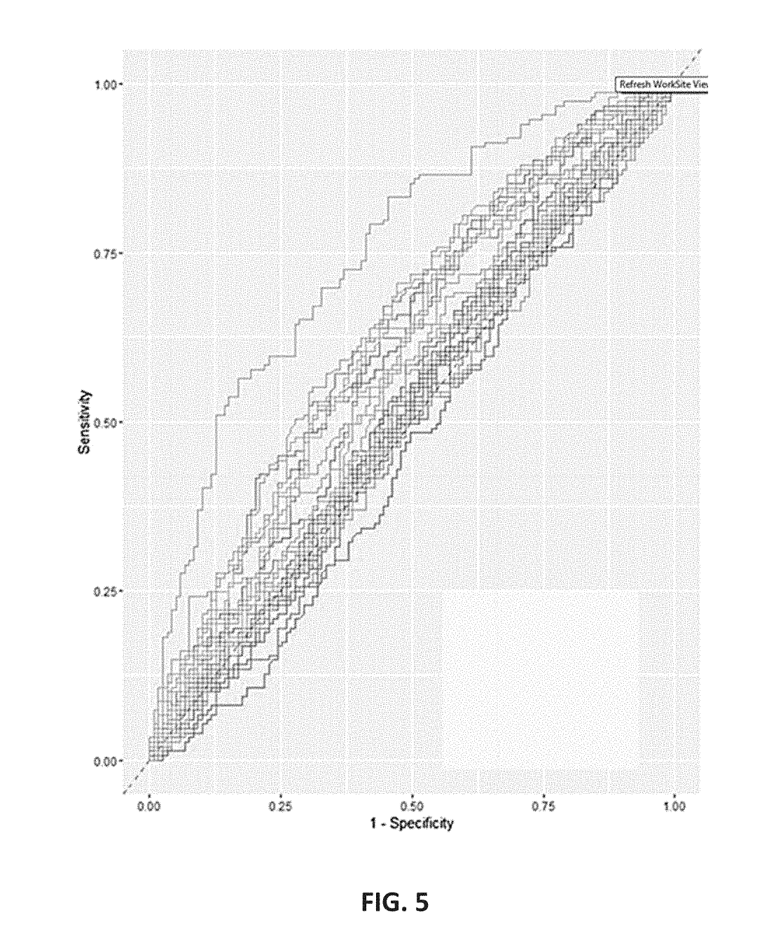

FIG. 6 is a waterfall plot of the autoantibody assay score across the validation cohort for prostate cancer monitoring panel.

FIG. 7A-B shows result interpretations for exemplary cancer profiling panels. FIG. 7A shows a result interpretation scale for a prostate cancer prognosis panel test score. The score is presented on a scale from 0 to 100 with a single cut point at 26. FIG. 7B shows a result interpretation scale for a prostate cancer monitoring panel test score. The score is presented on a scale from 0 to 100 with a higher risk cut point at 75 and a lower risk cut point at 35.

### DETAILED DESCRIPTION OF THE DISCLOSURE

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the invention may be described in the context of a single embodiment, the features may also be provided separately or in any suitable combination. Conversely, although the invention may be described herein in the context of separate embodiments for clarity, the invention may also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### Definitions

In this application, the use of the singular includes the plural unless specifically stated otherwise. It must be noted that, as used in the specification, the singular forms “a”, “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments”, “an embodiment,” “one embodiment” or “other embodiments” means that a particular feature, structure, or characteristic described in connection with the embodiments is included in at least some embodiments, but not necessarily all embodiments, of the inventions.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the invention, and vice versa. Furthermore, compositions of the invention can be used to achieve methods of the invention.

The term “about” in relation to a reference numerical value and its grammatical equivalents as used herein can include the numerical value itself and a range of values plus or minus 10% from that numerical value.

The term “approximately” means within an acceptable error range for the particular value as determined by one of ordinary skill in the art, which will depend in part on how the value is measured or determined, i.e., the limitations of the measurement system.

The terms “subject” or “patient”, used interchangeably herein refers to an animal, for example a human, to whom treatment, including prophylactic treatment, with the pharmaceutical composition according to the present invention, is provided. The term “subject” as used herein refers to human and non-human animals. In one embodiment, the subject is human. In another embodiment, the subject is an experimental animal or animal substitute as a disease model. The term does not denote a particular age or sex. Thus, adult and newborn subjects, as well as fetuses, whether male or female, are intended to be covered. Non-limiting examples of subjects include humans, dogs, cats, cows, goats, and mice. The term subject is further intended to include transgenic species. A subject suspected of having cancer refer to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a subject suspected of having cancer encompasses an individual who has received an initial diagnosis (e.g., a CT scan showing a mass or increased PSA level) but for whom the stage of cancer is not known. The term further includes people who once had cancer (e.g., an individual in remission). As used herein, a subject at risk for cancer or a subject having an increased risk of cancer refers to a subject with one or more risk factors for developing a specific cancer. Risk factors include, but are not limited to, gender, age, genetic predisposition, environmental exposure, previous incidents of cancer, preexisting non-cancer diseases, family history and lifestyle. In an embodiment, non-limiting exemplary risk factors for prostate cancer are age, elevated PSA, digital rectal exam (DRE), race, family history and urinary symptoms.

As used herein, the term “sample” refers to a biological sample obtained or derived from a source of interest, as described herein. In some embodiments, a source of interest comprises an organism, such as an animal or human. In some embodiments, a sample comprises nucleic acids or a set of nucleic acids (e.g., library) representing all or substantially of the nucleic acid sequences found in a source. In some embodiments, a sample comprises polypeptides or a set of amino acid sequences representing all or substantially of the polypeptide sequences found in a source. In some embodiments, a biological sample or source of the sample comprises biological tissue or fluid. In some embodiments, a biological sample or source of the sample may be or comprise blood, blood cells, ascites, tissue or fine needle biopsy samples, cell-containing body fluids, bone marrow, sputum, saliva, urine, tissue biopsy specimens, surgical specimens, other body fluids, secretions, and/or excretions, and/or cells therefrom. Such examples are not however to be construed as limiting the sample types applicable to the present invention. In some embodiments, a biological sample or source of the sample is or comprises cells obtained from an individual. In some embodiments, obtained cells are or include cells from an individual from whom the sample is obtained. The term “tissue” is intended to include intact cells, blood, blood preparations such as plasma and serum, bones, joints, cartilage, neuronal tissue (brain, spinal cord and neurons), muscles, smooth muscles, and organs.

The term “protein” used interchangeably with “polypeptide” or “peptide” refers to a polymer of amino acid residues linked by peptide bonds. The term “polypeptide” and “protein” can encompass a multimeric protein, e.g., a protein containing more than one domain or subunit. Proteins and peptides can be composed of linearly arranged amino acids linked by peptide bonds, whether produced biologically, recombinantly, or synthetically and whether composed of naturally occurring or non-naturally occurring amino acids, are included within this definition. The terms also include polypeptides that have co-translational (e.g., signal peptide cleavage) and post-translational modifications of the polypeptide, such as disulfide-bond formation, glycosylation, acetylation, phosphorylation, lipidation, proteolytic cleavage (e.g., cleavage by metalloproteases), and the like. Exemplary modifications which can be present in polypeptides of the present disclosure include, but are not limited to, acetylation, acylation, ADP-ribosylation, amidation, covalent attachment of flavin, covalent attachment of a heme moiety, covalent attachment of a polynucleotide or polynucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formulation, gamma-carboxylation, glycation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Furthermore, as used herein, a “polypeptide” or “peptide” refers to a protein that includes modifications, such as deletions, additions, and substitutions to the native sequence. These modifications can be deliberate, as through site-directed mutagenesis, or can be accidental, such as through mutations of hosts that produce the proteins, or errors due to PCR amplification or other recombinant DNA methods.

The terms “polynucleotide”, “nucleotide”, “nucleotide sequence”, “nucleic acid” and “oligonucleotide” are used interchangeably. They can refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “identical” and its grammatical equivalents as used herein or “sequence identity” in the context of two nucleic acid sequences or amino acid sequences of polypeptides refers to the residues in the two sequences which are the same when aligned for maximum correspondence over a specified comparison window. A comparison window refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein are at least 80%, 85%, 90%, 98% 99% or 100% identical to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can be 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or 100% identity 50%, 60%, 70%, 75%, 80%, 85%, 90%, 98%, 99% or 100% identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” and its grammatical equivalents as applied to nucleic acid or amino acid sequences mean that a nucleic acid or amino acid sequence comprises a sequence that has at least 90% sequence identity or more, at least 95%, at least 96%, at least 97%, at least 98% and at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

The term “substantially similar” or its grammatical equivalent as used herein means that a particular subject sequence varies from the sequence of the polypeptides or polynucleotides by one or more substitutions, deletions, or additions, but retains at least 50%, or higher, e.g., at least 55%, 60%, 65%, 70%, 75%, 80%, 85%, 90%, 95% or more, inclusive, of the function of the protein or the nucleotide as compared with the original protein or nucleotide. In determining polynucleotide sequences, all subject polynucleotide sequences capable of encoding substantially similar amino acid sequences are considered to be substantially similar to a reference polynucleotide sequence, regardless of differences in codon sequence.

As used herein, the term “autoantibody” refers to an antibody produced by a host (with or without immunization) and directed to a host antigen (e.g., a tumor antigen).

The term “humoral immune response” or “humoral response” as used herein refers to an immune response in which the body mounts a defense against microorganisms, viruses, and substrates that it recognizes as foreign and potentially harmful. The defense involves antibodies that are secreted by B cells. The B cells are activated when a specific antigen binds to the antibody, which is located on the surface of the B cells. Plasma B cells release antibodies specific for the antigen.

The term “epitope” as used herein refers to that portion of an antigen that makes contact with a particular antibody. When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The term “mimotope” can mimic the epitope of a protein or peptide. A mimotope can structurally similar to an antigen or epitope of an expressed protein, but is unrelated or weakly related at the protein sequence level.

The term “fragment” of a peptide, polypeptide or molecule as used herein refers to any contiguous polypeptide subset of the molecule. Accordingly, a “fragment” of a molecule, is meant to refer to any polypeptide subset of the molecule. In some embodiments, a polynucleotide or a polypeptide comprises a sequence with at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the polynucleotide or the polypeptide.

The term “epitope fragment” of a peptide, polypeptide, or molecule as used herein refers to any contiguous polypeptide subset at least 2 amino acids in length of the molecule that makes contact with an antibody.

The term “reactivity” or its grammatical equivalents means having substantially greater affinity for the polypeptide probes as described herein compared to affinity for other binding agents (e.g., autoantibody) to which no specific binding is exhibited. In some cases, this substantially greater affinity is at least 1.5-fold, at least 2-fold, at least 5-fold, at least 10-fold, at least 100-fold, at least 1000-fold, at least 10,000-fold, at least 100,000-fold, at least 10⁶-fold or more. It will be understood by those of skill in the art that although antibodies are highly specific, particular antibodies may be highly specific for more than one antigen (i.e., polypeptide probe). Various terms have been used in the art to describe this phenomenon, including the term “cross-reactivity”. The antigens to which the antibody cross-reacts may be structurally similar or may be structurally dissimilar.

The term “binding” or its grammatical equivalents as used herein refers to a direct association between two molecules, due to, for example, covalent, electrostatic, hydrophobic, and ionic and/or hydrogen-bond interactions under physiological conditions, and includes interactions such as salt bridges and water bridges, as well as any other conventional means of binding. The terms “specific binding”, “specifically binding” or its grammatical equivalents when used in reference to the interaction of an antibody and a protein or peptide means that the interaction is dependent upon the presence of a particular structure (i.e., the antigenic determinant or epitope) on the protein; in other words, the antibody is recognizing and binding to a specific protein structure rather than to proteins in general. For example, if an antibody is specific for epitope A, the presence of a protein containing epitope A (or free, unlabeled A) in a reaction containing labeled A and the antibody will reduce the amount of labeled A bound to the antibody. As used herein, the terms “non-specific binding”, “background binding” or its grammatical equivalents when used in reference to the interaction of an antibody and a protein or peptide refer to an interaction that is not dependent on the presence of a particular structure (i.e., the antibody is binding to proteins in general rather that a particular structure such as an epitope).

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an agent can be an oligomer of nucleic acids, amino acids, or carbohydrates including, but not limited to proteins, peptides, oligonucleotides, ribozymes, DNAzymes, glycoproteins, RNAi agents (e.g., siRNAs), lipoproteins, aptamers, and modifications and combinations thereof.

The term “detection” or its grammatical equivalents as used herein refers to methods by which the skilled artisan can estimate and/or determine the probability (a likelihood) of whether or not a patient is suffering from a given disease or condition. In the case of the present disclosure, diagnosis includes using the results of an assay for markers of the present disclosure, optionally together with other clinical characteristics, to arrive at a diagnosis (that is, the occurrence or nonoccurrence) of cancer, e.g., prostate cancer, for the subject from which a sample was obtained and assayed. That such a diagnosis is determined is not meant to imply that the diagnosis is 100% accurate. Many biomarkers are indicative of multiple conditions. The skilled clinician does not use biomarker results in an informational vacuum, but rather test results are used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold. Similarly, a prognostic risk signals a probability (a likelihood) that a given course or outcome will occur. A level or a change in level of a prognostic indicator, which in turn is associated with an increased probability of morbidity (e.g., aggressive cancer, such as a prostate cancer) is referred to as being indicative of an increased likelihood of an adverse outcome in a patient.

The term “aggressive prostate cancer” means prostate cancer that is poorly differentiated, having a Gleason score of 7 or above. The term “indolent prostate cancer” means prostate cancer having a Gleason score of 6. The Gleason score system is the most commonly used method for grading prostate cancer. Gleason Score (GS) is a system of grading prostate cancer cells based on how they look under a microscope. Gleason scores range from 2 to 10 and indicate how likely it is that a tumor will spread. A low Gleason score means the cancer cells are similar to normal prostate cells and are less likely to spread; a high Gleason score means the cancer cells are very different from normal and are more likely to spread. The grade of a tumor can depend on how abnormal the cancer cells look under a microscope and how quickly the tumor is likely to grow and spread. Grading systems are different for each type of cancer.

The term “active surveillance” is an active monitoring of patients with indolent cancer who are not receiving treatment. Patients with very low-risk prostate cancer (low grade, low stage, localized disease; Gleason score <6) are monitored carefully over time for signs of disease progression. Signs of disease progression triggers immediate active treatment.

The term “receiver operating characteristic” (ROC), or ROC curve, means a graphical plot that illustrates the performance of a binary classifier system as its discrimination threshold is varied. ROC curve analysis is often applied to measure the diagnostic accuracy of a biomarker. The analysis results in two gains: diagnostic accuracy of the biomarker and the optimal cut-point value. The ROC curve is a mapping of the sensitivity versus for all possible values of the cut-point between cases and controls. To measure the diagnostic ability of a biomarker, it is common to use summary measures such as the area under the ROC curve (AUC) and/or the partial area under the ROC curve (pAUC). A biomarker with AUC=1 discriminates individuals perfectly as diseased or healthy. Meanwhile, an AUC=0.5 means that there is no apparent distributional difference between the biomarker values of the two groups. ROC analysis provides two main outcomes: the diagnostic accuracy of the test and the optimal cut-point value for the test. Cut-points dichotomize the test values, so this provides the diagnosis (diseased or not). The identification of the cut-point value requires a simultaneous assessment of sensitivity and specificity. A cut-point is referred to as optimal when the point classifies most of the individuals correctly. AUC, sensitivity, and specificity values are useful for the evaluation of a marker; however, they do not specify “optimal” cut-points directly. The ROC curve is created by plotting the true positive rate (sensitivity) against the false positive rate (1—specificity) at various threshold settings. The true-positive rate is also known as sensitivity, recall or probability of detection in machine learning. The false-positive rate is also known as the fall-out or probability of false alarm and can be calculated as (1—specificity). The ROC curve is thus the sensitivity as a function of fall-out. In general, if the probability distributions for both detection and false alarm are known, the ROC curve can be generated by plotting the cumulative distribution function (area under the probability distribution from −∞ to the discrimination threshold) of the detection probability in the y-axis versus the cumulative distribution function of the false-alarm probability on the x-axis. ROC analysis provides tools to select possibly optimal models and to discard suboptimal ones independently from (and prior to specifying) the cost context or the class distribution. ROC analysis is related in a direct and natural way to cost/benefit analysis of diagnostic decision making. Area Under the ROC Curve (AUC) means a statistic to measure classifier performance, commonly used in machine learning applications that encapsulates both sensitivity and specificity of the classifier performance. In a ROC curve, the true positive rate (sensitivity) is plotted in function of the false positive rate (1—specificity) for different cut-off points of a parameter. Each point on the ROC curve represents a sensitivity/specificity pair corresponding to a particular decision threshold. The area under the ROC curve (AUC) is a measure of how well a parameter can distinguish between two diagnostic groups (diseased/normal).

The terms “positive predictive value” (PPV) and “negative predictive value” (NPV) are the proportions of positive and negative results in statistics and diagnostic tests that are true positive and true negative results, respectively. The PPV and NPV describe the performance of a diagnostic test or other statistical measure. A high result can be interpreted as indicating the accuracy of such a statistic. Although sometimes used synonymously, a negative predictive value generally refers to what is established by control groups, while a negative post-test probability rather refers to a probability for an individual. Still, if the individual's pre-test probability of the target condition is the same as the prevalence in the control group used to establish the negative predictive value, then the two are numerically equal.

### Overview

The compositions and methods of the present disclosure relate to compositions and methods for characterizing a cancer or screening for a cancer. Provided herein are tests which can be used to analyze a presence or absence of an antibody from a subject, such as a subject being tested or screened for a cancer. In one embodiment, an antibody is an autoantibody. In another embodiment, the test comprises a single antigen, thus detecting only an antibody that binds to that antigen. In another embodiment, a panel of antigens is constructed such that the panel tests for a presence of one or more antibodies which specifically bind to two or more antigens derived from proteins associated with a specific cancer, such as lung cancer, prostate cancer, or ovarian cancer. By detecting an antibody to a protein associated with a disease state, the compositions and methods provided herein allow for the characterization and diagnosis of a cancer.

A cancer is characterized for a subject using a composition or method disclosed herein. In one embodiment, a subject is an individual or patient. In one embodiment, a subject is a human. In another embodiment, a subject is a cancer patient. In yet another embodiment, a subject is a prostate cancer patient. In one embodiment, a subject exhibits no symptom of cancer, such as no symptoms of prostate cancer. In another embodiment, a subject has no detectable symptom of cancer, such as no detectable symptoms for prostate cancer. In yet another embodiment, a subject exhibits a symptom of cancer, such as a symptom for prostate cancer. In some embodiments, a subject has indolent cancer. In some embodiments, a subject has aggressive cancer. In some embodiments, the cancer detection platform as described herein improves risk assessment of prostate cancer in primary care setting to reduce unnecessary referrals to urologists. In some embodiments, the cancer prognosis platform as described herein differentiates indolent cancer from aggressive cancer to select appropriate patients for initial and repeat biopsy. In some embodiments, the cancer active monitoring platform is used to active monitor a subject with indolent cancer. In another embodiment, active monitoring avoids overtreatment. In one embodiment, a subject is a human. In another embodiment, a subject is an individual. In yet another embodiment, a subject is a patient, such as a cancer patient.

Characterizing a cancer, or screening for a cancer, can include detecting the cancer (including pre-symptomatic early stage detecting), risk assessment, determining the prognosis, diagnosis, discriminating indolent cancer from aggressive cancer, active monitoring (active surveillance) of the cancer, or determining the stage or progression of the cancer. In one embodiment, a diagnosis is prediction or likelihood an individual or subject has a disease or condition, such as prostate cancer. In one embodiment, the individual is an asymptomatic individual. In another embodiment, the individual is a symptomatic individual. In one embodiment, a prognosis is predicting or giving a likelihood of outcome of a disease or condition, such as an extent of malignancy of a cancer, a likelihood of survival, or expected life expectancy, such as in an individual with prostate cancer. In another embodiment, a prognosis is a prediction or likelihood analysis of cancer progression, cancer recurrence, or metastatic spread or relapse. In yet another embodiment, a prognosis differentiates indolent vs. aggressive disease to select appropriate patients for initial repeat biopsy. In one embodiment, cancer active surveillance is an active monitoring of a patient with indolent cancer who is not receiving treatment. In one embodiment, a therapy is selected based on an outcome of determining a binding of one or more antibodies from a sample from a subject to a plurality of polypeptide probe as described herein. In one embodiment, a serum-based non-PSA test as described herein identifies an appropriate treatment or treatment efficacy for a cancer. In some embodiments, a treatment is modified based on the results of the serum-based non-PSA test. In another embodiment, a treatment regimen is selected based on the results of the serum-based non-PSA test. In yet another embodiment, a treatment regimen is discontinued or not selected selected based on the results of the serum-based non-PSA test. In some embodiment, cancer treatment monitoring allows patient selection and monitoring to help determine therapeutic effectiveness.

In one embodiment a treatment regimen or therapeutic agent is selected based on the presence or absence of an autoantibody that binds to polypeptide probes described herein. In one embodiment the autoantibody is a human autoantibody. In one embodiment a treatment regimen or therapeutic agent is excluded based on the presence or absence of an autoantibody that binds to polypeptide probes described herein. In one embodiment the autoantibody is a human autoantibody. In yet another embodiment, characterizing or screening for a cancer is detecting the cancer, such as pre-symptomatic early stage detecting, or differentiating indolent vs. aggressive cancer. In yet another embodiment, a patient with indolent cancer who is not receiving treatment is monitored under active surveillance. In one embodiment, characterizing a cancer is determining the stage or progression of the cancer, such as early-stage, late-stage or advanced stage of cancer. Characterizing or screening for a cancer can also be determining the likelihood or possibility an individual has a cancer. Characterizing or screening for a cancer can also be identification of a cancer, such as determining whether expression of one or more antibodies is indicative of the cancer.

In one embodiment, an antigen panel is used to detect a presence of one or more antibodies to one or more proteins, antigens, mimotopes, or epitopes. In one embodiment, one or more polypeptide probes described herein is a protein or fragment thereof. In another embodiment, one or more polypeptide probes described herein comprises an antigen, mimotope, or epitope.

A cancer can also be characterized by determining a presence or absence, or level, of one or more antibodies in a sample. In one embodiment, a sample is obtained from a subject. The subject can be a mammal, including, but not limited to, humans, non-human primates, rodents, and the like. In another embodiment, a sample is a biological fluid. The biological fluid can be, but not limited to, peripheral blood, sera, or plasma. The sample can be ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, or bronchopulmonary aspirates.

In one embodiment, the level, presence, or absence of an antibody can be determined by detecting the binding of one or more antibodies to a polypeptide probe. In one embodiment, an antibody is an autoantibody. An autoantibody refers to an antibody produced by a host (with or without immunization) and directed to a host antigen (such as a tumor antigen). Tumor-associated antigens recognized by humoral effectors of the immune system are an attractive target for diagnostic and therapeutic approaches to human cancer.

The binding of an antibody with a polypeptide probe can be specific, such that the interaction of the autoantibody with the polypeptide probe is dependent upon a presence of a particular structure (e.g., the antigenic determinant or epitope) of the polypeptide probe. Antigenic determinates or epitopes can comprise amino acids in linear or non-linear sequence in a polypeptide probe and can also comprise one or more amino acids which are in proximity to each other via protein folding (e.g., conformational epitopes). Thus, a single polypeptide or protein can potentially be bound by multiple antibodies which recognize different epitopes. In some instances, known epitopes of a particular polypeptide can be used as a probe to detect for a presence, absence or level of autoantibodies which bind a particular epitope

### Polypeptide Probes

Provided herein is a composition and method for detecting one or more antibodies in a sample using one or more polypeptide probes or a plurality of polypeptide probes. In one embodiment, the plurality of polypeptide probes comprises a polypeptide, a mimotope of the polypeptide or an epitope fragment of the polypeptide. Polypeptide is used in its broadest sense and can include a sequence of subunit amino acids, amino acid analogs, peptidomimetics, epitope fragments or mimotopes. The subunits can be linked by peptide bonds. The polypeptides can be naturally occurring, processed forms of naturally occurring polypeptides (such as by enzymatic digestion), chemically synthesized or recombinantly expressed. The polypeptides for use in the methods of the present disclosure can be chemically synthesized using standard techniques. The polypeptides can comprise D-amino acids (which are resistant to L-amino acid-specific proteases), a combination of D- and L-amino acids, β-amino acids, or various other designer or non-naturally occurring amino acids (e.g., β-methyl amino acids, Cα-methyl amino acids, and Nα-methyl amino acids, etc.) to convey special properties.

The polypeptides disclosed herein can comprise synthetic amino acids in place of one or more naturally-occurring amino acids. Synthetic amino acids can include ornithine for lysine, and norleucine for leucine or isoleucine. Such synthetic amino acids are known in the art, and non-limiting example include aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides described herein in an engineered cell can be associated with post-translational modifications of one or more amino acids of the polypeptide constructs. Non-limiting examples of post-translational modifications include phosphorylation, acylation including acetylation and formylation, glycosylation (including N-linked and O-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

In addition, the polypeptides can have peptidomimetic bonds, such as ester bonds, to prepare polypeptides with novel properties. For example, a polypeptide can be generated by incorporating a reduced peptide bond, i.e., R₁—CH₂—NH—R₂, where R₁ and R₂ are amino acid residues or sequences. A reduced peptide bond can be introduced as a dipeptide subunit. Such a polypeptide can be resistant to protease activity and can possess an extended half-life in vivo. A polypeptide can also include a peptoid (N-substituted glycines), in which the one or more side chains are appended to nitrogen atoms along the molecule's backbone, rather than to the α-carbons, as in amino acids. Polypeptide and peptide are intended to be used interchangeably throughout this application, i.e., where the term peptide is used, it can also include polypeptide and where the term polypeptides is used, it can also include peptide.

In some embodiments, a polypeptide probe can be an antigen identified through serologic identification of antigens, for example, by recombinant expression cloning (SEREX), such as described by Kim et al., Biotech. Lett. (2004); 26: 585-588. Generally, in this method, an antigen can be identified by screening expression cDNA libraries from human solid tumors with sera of autologous patients. This type of screening of a cDNA expression library by conventional methods typically requires the preparation of a large number of membrane filters blotted with bacteriophage plaques that are then searched with a specific probe. In the case of the SEREX experiments, the screening is performed using sera from cancer patients, which can be in very limited quantities.

In some embodiments, a polypeptide probe for detecting an antibody can also be identified by phage display technology, which can be based on the insertion of nucleotide sequences into genes encoding for various capsid proteins of T7 phage, resulting in a heterogeneous mixture of phages, each displaying the different peptide sequence encoded by a corresponding insert. The platform of phage-epitope microarrays is capable of detecting over 2300 phage clones in one microarray using only microliters of sera. Highly parallel assays using different patient samples are easily compared using protein microarray technology that allows for the molecular classification of cancer based on epitomic profiles (akin to molecular profiles based on gene expression). A physical link between a displayed fusion protein and DNA encoded for it make this phage target selectable. The phage target can express or display a polypeptide probe, which can be used to detect antibodies that are produced by a subject (autoantibodies), which can then be used to detect or characterize a cancer. A polypeptide probe can be displayed by a phage and used to detect an antibody from a sample obtained from a subject. In one embodiment, an antibody is an autoantibody. The present disclosure is not limited by the nature of the peptide display system used.

In some embodiments, the methods described herein detect antibodies that are produced by patients in reaction to proteins expressed in their tumors. These markers find use as diagnostic or prognostic biomarkers and therapeutic targets. In another embodiment, these markers can be used to actively monitor patients with indolent cancer who are not receiving treatment. In some embodiments, the methods described herein employ pattern recognition of multiple markers as a diagnostic or a prognostic rather than any single marker. In another embodiment, active monitoring employs pattern recognition of multiple markers rather than any single marker. Features of the approach include acknowledging the heterogeneous nature of any specific kind of cancer, and using specialized bioinformatics techniques to interpret the results. In some embodiments, the methods described herein employ the recognition of a pattern of immunologic response as a diagnostic or prognostic strategy.

In some embodiments, experiments conducted during the course of development of the present disclosure resulted in the detection of a serum reaction with large numbers of epitopes using a highly parallel phage display assay on protein microarrays. Once the chosen epitope markers are spotted on the final version of the array, serum from both cancer patients and controls are tested. In some embodiments, the results of the reaction of the sera with the various subjects are used to train a machine learning device to build a predictor and further to test unknown samples.

In some embodiments, the methods of the phage microarray profiling utilize fluorescent probes and laser scanner, resulting in high sensitivity and the detection of very small signal differences. In addition, the methods of the phage microarray profiling allow for detection at the protein expression level rather than cDNA level as compared to cDNA or oligo arrays. In some embodiments, the methods of the phage microarray profiling utilize an analytical approach rather that a visual assessment, which results in greater consistency and reproducibility. Further, due to the high sensitivity of this technique, low amounts (e.g., only 1-2 μl) of serum samples may be used. The methods of the phage microarray profiling are rapid and allow for the analysis of protein-protein interactions.

In other embodiments, the present disclosure provides a phage array profile map comprising protein array profiles of cancers of various stages or prognoses (e.g., likelihood of future metastasis and risk assessment). Such maps can be used for comparison with patient samples. Any suitable method may be utilized, including but not limited to, by computer comparison of digitized data. The comparison data is used to provide diagnoses and/or prognoses to patients. The comparison data can also be used to provide active monitoring of patients with indolent cancer who are not receiving treatment. In some embodiments, the comparison data can also be used for patient selection and monitoring to help determine therapeutic effectiveness.

In one embodiment, a polypeptide probe disclosed herein is attached to a substrate (e.g., glass slide chip or nanowell chip). A polypeptide probe can be directly or indirectly attached to the substrate. In one embodiment, a polypeptide probe is attached to a substrate via a phage. The substrate can be any physically separable solid to which a polypeptide probe can be directly or indirectly attached including, but not limited to, surfaces provided by microarrays and wells, particles such as beads, columns, optical fibers, wipes, glass and modified or functionalized glass, quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polypropylene, polyethylene, polybutylene, polyurethanes, TEFLON™, etc.), polysaccharides, nylon or nitrocellulose, resins, silica or silica-based materials including silicon and modified silicon, carbon, metals, inorganic glasses, plastics, ceramics, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers.

The polypeptide probe can bound to a planar surface or to a particle, such as a bead or microsphere. In one embodiment, the polypeptide probe is attached to a bead. The bead can be a polystyrene, brominated polystyrene, polyacrylic acid, polyacrylonitrile, polyacrylamide, polyacrolein, polydimethylsiloxane, polybutadiene, polyisoprene, polyurethane, polyvinyl acetate, polyvinylchloride, polyvinylpyridine, polyvinylbenzylchloride, polyvinyltoluene, polyvinylidene chloride, polydivinylbenzene, polyglycidylmethacrylate, polymethylmethacrylate, or copolymers, blends, composites, or combination thereof. The bead can have a diameter of between about 1 nm-1000 μm, 1 nm-500 μm, 5 nm-500 μm, or 10 nm-100 μm. In one embodiment, the bead has a diameter of between about 10 nm and 100 μm. In yet another embodiment, the bead has a diameter of less than about 1000 μm, 500 μm, 400 μm, 300 μm, 200 μm, or 100 μm.

In one embodiment, the bead is labeled or stained with more than one dye, such as at least 2, 3, 4, 5, 6, 7, 8, 9 or 10 different dyes. In one embodiment, the bead is labeled or stained with two dyes. In another embodiment, the two dyes are hydrophobic. In another embodiment, the two dyes are fluorescent dyes, such as squaric acid-based dyes. In yet another embodiment, the squaric acid-based dyes are selected from cyclobutenedione derivatives, symmetrical and unsymmetrical squaraines, substituted cephalosporin compounds, fluorinated squaraine compositions, alkylalkoxy squaraines, or squarylium compounds. In another embodiment, the squaric acid-based dyes are selected from a red fluorescent dye and an orange fluorescent dye, such as the red fluorescent dye comprising 1,3-bis(1,3-dihydro-1,3,3-trimethyl-2H-indol-2-ylidene)methyl]-2,4-dihydro xycyclobutenediylium, bis(inner salt) and the orange fluorescent dye comprising 2-(3,5-dimethylpyrrol-2-yl)-4-(3,5-dimethyl-2H-pyrrol-2-ylidene)-3-hydroxy-2-cyclobuten-1-one.

In one embodiment, the substrate is coated using passive or chemically-derivatized coatings with any number of materials, including polymers, such as dextrans, acrylamides, gelatins or agarose. Such coatings can facilitate the use of the array with a biological sample.

In some embodiments, a polypeptide probe can be a fragment or portion of a larger protein. A fragment can range in size from two amino acid residues to the entire amino acid sequence minus one amino acid. In one embodiment, a polypeptide probe is a fragment of an untranslated region (UTR) of a protein, such as a fragment that is encoded by a nucleic sequence that is a UTR region of a gene, such as the 5′ or 3′ UTR of a gene. The fragment can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids in size. In one embodiment, the fragment is less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids in size.

A polypeptide probe useful in the compositions and methods herein, regardless of size, is capable of specific interaction with an antibody, such as an autoantibody. A polypeptide probe can comprise a protein or fragment thereof encoded by a gene or mRNA (or a fragment thereof). A polypeptide probe can comprise a protein or fragment thereof that is the result of the mis-translation of all or a portion of a gene or mRNA. For example, the protein or fragment thereof can be the translation of a region upstream or downstream of a coding sequence, such as a UTR region, of an mRNA. In another example, the protein or fragment thereof can be the result of translating a gene or mRNA in the wrong frame, the wrong orientation, or both.

In one embodiment, a polypeptide probe can comprise a peptide sequence or a fragment thereof, or a peptide sequence encoded by a nucleic acid or a complement thereof, such as those listed in Table 1. A number of markers are disclosed in Table 1. The markers listed in Table 1 were discovered using phage-display technology. The marker names are based on the identity of the cDNA sequence inserted into a phage display vector. However, the marker names do not necessarily identify the peptide sequence displayed by a phage, at least because: the cDNA sequence may not be from a coding sequence (e.g., it may be from an untranslated region of an mRNA); the cDNA sequence may not have been inserted in the natural coding frame (e.g., it may be inserted in the opposite orientation, the wrong coding frame, or both); the cDNA sequence may contain one or more mutations (e.g., substitutions, deletions, insertions, recombinations) in comparison to the most closely matched, endogenous mRNA; or a combination thereof. Peptide sequences that do not correspond to an endogenous protein sequence likely represent mimotopes.

TABLE 1

| | SEQ ID | | SEQ ID | |
|---|---|---|---|---|
| | NO | Peptide | NO | |
| Marker | (Peptide) | sequence | (cDNA) | cDNA sequence |
| hnRNPA1 | 1 | QSVFKFWT | 2 | CAATCAGTCTTCAAATTTTGGACCCATGAAGGGAGG |
| | | HEGRKFWR | | AAATTTTGGAGGCAGAAGCTCTGGCCCCTATGGCGG |
| | | QKLWPLWR | | TGGAGGCCAATACTTTGCAAAACCACGAAACCAAGG |
| | | WRPILCKT | | TGGCTATGGCGGTTCCAGCAGCAGCAGTAGCTATGG |
| | | TKPRWLWR | | CAGTGGCAGAAGATTTTAATTAGGAAACAAAGCTTT |
| | | FQQQQ | | |
| KDELR1 | 3 | GQMGVGRF | 4 | GGGCAGATGGGTGTCGGCAGATTTAGTGTTTGGCAA |
| | | SVWQPVGL | | CCAGTGGGGCTGGGGGTGGGATCTGGGAGGGAGCCG |
| | | GVGSGREP | | AGGGGCCTGGGGAAGGGAAAAGATCTTGGACCCTGC |
| | | RGLGKGKD | | CCCGGCCCATAGGACACTCAAAAACACTTTATAAAA |
| | | LGPCPGP | | ATTGGGG |
| SMAD3 | 5 | ILYKNSSK | 6 | ATTTTATACAAAAACAGCAGCAAAGAATAAAGTACC |
| | | E | | AATTGAAGGTGGCAGTTCTGTCTACTGTGTTCTGCA |
| | | | | AAAGGCTGGGAAAAGAAGAGGACTCTTTTTTCTTTT |
| | | | | TTCCAGTTGAAATAATTTAGACTAGTTTTCTCCCTA |
| | | | | ATTGCTGCTGGGAGCAGTACTTGTTACACAATACAA |
| | | | | CTGCTATAGTTGTCACCGGTGATCCTCTACCGGGAG |
| | | | | CTAGATCATCTTATATCCCAGGGGGAAGATGACAGA |
| | | | | CCTCTTTGTACCAAGCCTGCAATTTTCAAAGGTGTA |
| | | | | AAAATATTTTAATAGCCCTTTTCATTTGCCTATGTT |
| | | | | GGCCTGAGATGTTTCCCTTCTGGTGAATTTGCTTCT |
| | | | | GTAAATTTCAGCTTGCAGAAGTGCTGGCAGGTATCT |
| | | | | CTTCAACAAAACCTGGAAAA |
| FKBP4 | 7 | NSEEKLEQ | 8 | AATTCAGAAGAGAAGCTGGAACAGAGCACCATAGTG |
| | | STIVKERG | | AAAGAGCGGGGCACTGTGTACTTCAAGGAAGGTAAA |
| | | TVYFKEGK | | TACAAGCAAGCTTTACTACAGTATAAGAAGATCGTG |
| | | YKQALLQY | | TCTTGGCTGGAATATGAGTCTAGTTTTTCCAATGAG |
| | | KKIVSWLE | | GAAGCACAGAAAGCACAGGCCCTTCGACTGGCCTCT |
| | | YESSFSNE | | CACCTCAACCTGGCCATGTGTCATCTGAAACTACAG |
| | | EAQKAQAL | | GCCTTCTCTGCTGCCATTGAAAGCTGTAACAAGGCC |
| | | RLASHLNL | | CTAGAACTGGACAGCAACAACGAGAAGGGCCTCTTC |
| | | AMCHLKLQ | | CGCCGGGGAGAGGCCCACCTGGCCGT |
| | | AFSAAIES | | |
| | | CNKALELD | | |
| | | SNNEKGLF | | |
| | | RRGEAHLA | | |
| TXNRD1 | 9 | LTVPHRAL | 10 | CTGACCGTCCCCCACAGGGCTCTGCCTCACGTCCTC |
| | | PHVLISFG | | ATCTCATTTGGCTGTGTAAAGAAATGGGAAAAGGGA |
| | | CVKKWEKG | | AAAGGAGAGAGCAATTGAGGCAGTTGACCATATTCA |
| | | KGESN | | GTTTTATTTATTTATTTTTAATTTGTTTTTTTCTCC |
| | | | | AAGTCCACCAGTCTCTGAAATTAGAACAGTAGGCGG |
| | | | | TATGAGATAATCAGGCCTAATCATGTTGTGATTCTC |
| | | | | TTTTCTTAGTGGAGTGGAATGTTCTATCCCCACAAG |
| | | | | AAGGATTATATCTTATAGACTTGTCTTGTTCAGATT |
| | | | | CTGTATTTACCCATTTTATTGAAACATATACTAAGT |
| | | | | TCCATGTATTTTTGTTACAAA |
| RGS1 | 11 | GLTSKLSR | 12 | GGATTAACATCAAAGTTATCCAGGCGCAGAGTTGAA |
| | | RRVEEA | | GAAGCATAAGCAAGACAAAAACAGAGAGACCGCAGA |
| | | | | AGGAGGAAGATACTGTGGTACTGTCATAAAAAACAG |
| | | | | TGGAGCTCTGTATTAGAAAGCCCCTCAGAACTGGGA |
| | | | | AGGCCAGGTAACTCTAGTTACACAGAAACTGTGACT |
| | | | | AAAGTCTATGAAACTGATTACAACAGACTGTAAGAA |
| | | | | TCAAAGTCAACTGACATCTATGCTACATATTATTAT |
| | | | | ATAGTTTGTACTGAGCTATTGAAGTCCCAT |
| FOXA1 | 13 | ARRLMGFS | 14 | GCCAGGCGTTTAATGGGGTTTAGTAGGGTGGGGTTA |
| | | RVGLFSLM | | TTTTCGTTAATGTTAGTAAGGGTGGGGAAGCGAGGT |
| | | LVRVGKRG | | TGACCTGTTAGGGTGAGAAGAATTATTCGAGTGCTA |
| | | | | TAGGCGCTTGTCAGGGAGGTAGCGATGAGAGTAATA |
| | | | | GATAGGTGATATACATGATACATTCTCAAGAGTTGC |
| | | | | TTGACCGAAAGTTACAAGGACCCCAACCCCTTTGTC |
| | | | | CTCTCTACCCACAGATGGCCCTGGGAATCAATTCCT |
| | | | | CAGGAATTGCCCTCAAGAACTCTGCTTCTTGCTTTG |
| | | | | CAGAGTGCCATGGTCATGTCATTCTGAGGTCACATA |
| | | | | A |
| EEF1A1 | 15 | EKIDRRSG | 16 | GAAAAGATTGATCGCCGTTCTGGTAAAAAGCTGGAA |
| | | KKLEDGPK | | GATGGCCCTAAATTCTTGAAGTCTGGTGATGCTGCC |
| | | FLKSGDAA | | ATTGTTGATATGGTTCCTGGCAAGCCCATGTGTGTT |
| | | IVDMVPGK | | GAGAGCTTCTCAGACTATCCACCTTTGGGTCGCTTT |
| | | PMCVESFS | | GCTGTTCGTGATATGAGACAGACAGTTGCGGTGGGT |
| | | DYPPLGRF | | GTCATCAAAGCAGTGGACAAGAAGGCTGCTGGAGCT |
| | | AVRDMRQT | | GGCAAGGTCACCAAGTCTGCCCAGAAAGCTCAGAAG |
| | | VAVGVIKA | | GCTAA |
| | | VDKKAAGA | | |
| | | GKVTKSAQ | | |
| | | KAQKA | | |
| Hsp90AB1 | 17 | EVAAEEPN | 18 | GAAGTGGCAGCAGAGGAACCCAATGCTGCAGTTCCT |
| | | AAVPDEIP | | GATGAGATCCCCCCTCTCGAGGGCGATGAGGATGCG |
| | | PLEGDEDA | | TCTCGCATGGAAGAAGTCGATTAGGTTAGGAGTTCA |
| | | SRMEEVD | | TAGTTGGAAAACTTGTGCCCTTGTATAGTGTCCCCA |
| | | | | TGGGCTCCCACTGCAGCCTCGAGTGCCCCTGTCCCA |
| | | | | CCTGGCTCCCCCTGCTGGTGTCTAGTGTTTTTTTCC |
| | | | | CTCTCCTGTCCTTGTGTTGAAGGCAGTAAACTAAGG |
| | | | | GTGTCAAGCCCCATTCCCTCTCTACTCTTGACAGCA |
| | | | | GGATTGGATGTTGTGTATTGTGGTTTATTTTATTTT |
| | | | | CTTCATTTTGTTCTGAAATTAAAGTATGCAAAATAA |
| CRKL:S100A8 | 19 | LNSIIDVY | 20 | TTGAACTCTATCATCGACGTCTACCACAAGTACTCC |
| | | HKYSLIKG | | CTGATAAAGGGGAATTTCCATGCCGTCTACAGGGAT |
| | | NFHAVYRD | | GACCTGAAGAAATTGCTAGAGACCGAGTGTCCTCAG |
| | | DLKKLLET | | TATATCAGGAAAAAGGGTGCAGACGTCTGGTTCAAA |
| | | ECPQYIRK | | GAGTTGGATATCAACACTGATGGTGCAGTTAACTTC |
| | | KGADVWFK | | CAGGAGTTCCTCATTCTGGTGATAAAGATGGGCGTG |
| | | ELDINTDG | | GCAGCCCACAAAAAAAGCCATGAAGAAAGCCACAAA |
| | | AVNFQEFL | | GAGTAGCTGAGTTACTGGGCCCAGAGGCTGGGCCCC |
| | | ILVIKMGV | | TGGACATGTAAAAATAATTTTAAAAGCTTACACAGA |
| | | AAHKKSHE | | AAATATTATTGCCTGAAGTTTATGATCTTTAAGTTA |
| | | ESHKE | | CAGGTCAAAAGAGTTTTATGTTGTTGTTGTTGTTGT |
| | | | | TGTTGTTTTAAAACACACAGTGAAGACCGTCTAAGA |
| | | | | AGCAAGGCCACTGTTCCTCCTGTAGGGACAACGATC |
| | | | | CTGTCTCAGAGACCTGTCCTTCTGCTCCTTTAGAAA |
| | | | | ACAGAACCAGCACAGCGTCCACACCGACAGCAGTTA |
| | | | | CTAGGGGCATAGTCCTCCGGTCGCAGAGGGTGGAGT |
| | | | | TGAGGCAGAGCTCAGTGGTGTCCACAGAAAGCATTA |
| | | | | GTCTTAGGTACTGTGTTAAAACAATCAAGAATTCGG |
| | | | | ATCCCCGAGCATCACACCTGACTGGATCGAACCCCG |
| | | | | NCTCCAAG |
| NKX3-1 | 21 | RREGKSRW | 22 | CGGAGAGAGGGAAAATCAAGGTGGTATTTTCCAGCA |
| | | YFPALCMI | | CTTTGTATGATTTTGGATGAGTTGTACACCCAAGGA |
| | | LDELYTQG | | TTCTGTTCTGCAACTCCATCCTCCTGTGTCACTGAA |
| | | FCSATPSS | | TATCAACTCTGAAAGAGCAAGCTTGCGGCCGCACTC |
| | | CVTEYQL | | GAGTAACTAGTTAACCCCTTGGGGCCTCTAAACGGG |
| | | | | TCTTGAGGGGTTAACTAGTTACTCGAGTGCGGCCGC |
| | | | | AAGCTTGCTCTTTCAGAGTTGATATTCAGTGACACA |
| | | | | GGAGGATGGAGTTGCAGAACAGAATCCTTGGGTGTA |
| | | | | CAACTCATCCAAAATCATACAAAGTGCTGGAAAATA |
| | | | | CCACTTGATTTTCCCTCTCT |
| ARF6 | 23 | QKCRLQRQ | 24 | CAGAAATGTAGACTGCAAAGGCAGTATACAGGAAAA |
| | | YTGKGGVG | | GGTGGAGTGGGTTTTGTTTATGAGGGTGTCTGAAAA |
| | | FVYEGV | | CTAAAATTGAGCGGGATATCATGGTATAGTTGGACA |
| | | | | GTATTGGTCCTTCACACTTTGGCCATATTGTATAAT |
| | | | | GGAGCTTTTACCAAAGATGTATGAGAAGTGTAAGAC |
| | | | | TATAAAAAAATGAACTATTCAAAGTAAAACTCTTAA |
| | | | | CAAACATTTTACTTAAAGCAGATGCAAAAGGGTATT |
| | | | | CTCATGTAGGCTCCTGTTGGTGCAGAGGGATTTTTT |
| | | | | TGATTTCAGGATACAACTAAAGTACGAAGTTCTCAG |
| | | | | TTTCACTTTAGTAGAAAGAGCTCTAGAAATGAGGCT |
| | | | | GATAAACACATCTAAGAACACTGGTTGCTTTCTAAA |
| | | | | ATTTCCAAAGCTCCACCATAAATGTAATTTTTAGTG |
| | | | | TTTCAAATGATTGCATTTTAAAGTATATAAATATGG |
| | | | | GTTATCCAATATCAATGCTATAGTAACATCCTGAAA |
| | | | | CAAAACAAGCACAAAGGTATAAATGCCTAAACTGGA |
| | | | | GGAAGCTTG |
| AURKAIP1 | 25 | GRPPWPVS | 26 | GGCCGCCCGCCTTGGCCCGTCTCTGGAGTGCTGGGC |
| | | GVLGSRVC | | AGCCGGGTCTGCGGGCCCCTTTACAGCACATCGCCG |
| | | GPLYSTSP | | GCCGGCCCAGGTAGGGCGGCCTCTCTCCCTCGCAAG |
| | | AGPGRAAS | | GGGGCCCAGCTGGAGCTGGAGGAGATGCTGGTCCCC |
| | | LPRKGAQL | | AGGAAGATGTCCGTCAGCCCCCTGGAGAGCTGGCTC |
| | | ELEEMLVP | | ACGGCCCGCTGCTTCCTGCCCAGACTGGATACCGGG |
| | | RKMSVSPL | | ACCGCAGGGACTGTGGCTCCACCGCAATCCTACCAG |
| | | ESWLTARC | | TGTCCGCCCAGCCAGATAGGGGAAGGGGCCGAGCAG |
| | | FLPRLDTG | | GGGGATGAAGGCGTCGCGGATGCGCCTCAAATTCAG |
| | | TAGTVAPP | | TGCAAAAACGTGCTGAAGATCCGCCGGCGGAAGATG |
| | | QSYQCPPS | | AA |
| | | QIGEGAEQ | | |
| | | GDEGVADA | | |
| | | PQIQCKNV | | |
| | | LKIRRRKM | | |
| CSNK2A2 | 27 | SSCSEYNV | 28 | TCATCCTGCTCGGAGTACAATGTTCGTGTAGCCTCA |
| | | RVASRYFK | | AGGTACTTCAAGGGACCAGAGCTCCTCGTGGACTAT |
| | | GPELLVDY | | CAGATGTATGATTATAGCTTGGACATGTGGAGTTTG |
| | | QMYDYSLD | | GGCTGTATGTTAGCAAGCATGATCTTTCGAAGGGAA |
| | | MWSLGCML | | CCATTCTTCCATGGACAGGACAACTATGACCAGCTT |
| | | ASMIFRRE | | GTTCGCATTGCCAAGGTTCTGGGTACAGAAGAACTG |
| | | PFFHGQDN | | TATGGGTATCTGAAGAAGTATCACATAGACCTAGAT |
| | | YDQLVRIA | | CCACACTTCAACGATATCCTGGGACAACATTCACGG |
| | | KVLGTEEL | | AAACGCTGGGAAAACTTTATCCATAGTGAGAACAGA |
| | | YGYLKKYH | | CACCTTGTCAGCCCTGAGGCCCTAGATCTTCTGGAC |
| | | IDLDPHFN | | AAACTTCTGCGATACGACCATCAACAGAGACTGACT |
| | | DILGQHSR | | GCCAAAGAGGCCATGGAGCACCCATACTTCTACCCT |
| | | KRWENFIH | | GTGGTGAAGGAGCAGTCCCAGCCTTGTGCAGACAAT |
| | | SENRHLVS | | GCTGTGCTTTCCAGTGGTCTCACGGCAGCACGATGA |
| | | PEALDLLD | | AGACTGGAAAGCGACGGGT |
| | | KLLRYDHQ | | |
| | | QRLTAKEA | | |
| | | MEHPYFYP | | |
| | | VVKEQSQP | | |
| | | CADNAVLS | | |
| | | SGLTAAR | | |
| UTR-BMI1 | 29 | GGRGGGGG | 30 | GGAGGTCGAGGCGGAGGCGGAGGAGGAGGAGGCCGA |
| | | GGGRGAGG | | GGCGCCGGAGGAGGCCGAGGCGCCGGAGCAGGAGGA |
| | | GRGAGAGG | | GGCCGGCCGGAGGCGGCATGAGACGAGCGTGGCGGC |
| | | GRPEAA | | CGCGGCTGCTCGGGGCCGCGCTGGTTGCCCATTGAC |
| | | | | AGCGGCGTCTGCAGCTCGCTTCAAGATGGCCGCTTG |
| | | | | GCTCGCATTCATTTTCTGCTGAACGACTTTTAACTT |
| | | | | TCATTGTCTTTTCCGCCCGCTTCGATCGCCTCGCGC |
| | | | | CGGCTGCTCTTTCCGGGATTTTTTATCAAGCAGAAA |
| | | | | TGCGTCGAACAACGAGAATCAAGATCACTGAGCTAA |
| | | | | ATCCCCACCTGATGTGTGTGCTTTGTGGAGGGTACT |
| | | | | TCATTGATGCCACAACCATAATAGAATGTCTACATT |
| | | | | CCTTCTGTAAAACGTGTATTGTTCGTTACCTGGAGA |
| | | | | CCAGCAAGTATTGTCCTATTTGTGATGTCCAAGTTC |
| | | | | ACAAGACCAGACCACTACTGAATATAAGGTCAGATA |
| | | | | AAACTCTCCAAGATATTGTATACAAATTAGTTCCAG |
| | | | | GGCTTTTCAAAAATGAAATGAAGAGAAGAAGGGATT |
| | | | | TTTATGCAGCTCATCCTTCTGCTGATGCTGCCAATG |
| | | | | GCTCTAATGAAGATAGAGGAGAGGTTGCAGATGAAG |
| | | | | ATAAGAGAATTATAACTGATGATGAGATAATAAGCT |
| | | | | TGCGGCCGCCCTCGAG |
| CEP164 | 31 | ARDLGEAA | 32 | GCTAGGGACCTCGGAGAAGCTGCTCTGGTAGCTGAG |
| | | LVAERKRE | | AGAAAGAGGGAGGAGGTGACAGATGTGATGGCCTCT |
| | | EVTDVMAS | | GTGCATCCTCTGTCACTTCCGCGCCTCCTCTCTCCC |
| | | VHPLSLPR | | CTCGCCATGCTCTCCTCTTCCTTCCCAGTGAGCAGC |
| | | LLSPLAML | | TCCGGCTCCTACAGCACTCCCATTCGCAAGTCCCTG |
| | | SSSFPVSS | | AGGCGGGCAGCACCTCCTTTCAGGGCATAATTGAGG |
| | | SGSYSTPI | | CCAACCGGAGGTGGCTGGAACGTGTCAAGAATGACC |
| | | RKSLRRAA | | CCAGGTTACCTCTCTTCTCGTCAACACCCAAGCCAA |
| | | PPFRA | | AAGCTACTTTGAGCCTCCTGCATGCTGGGCCTTGAT |
| | | | | GAGCACAATCAGATGTGAAGGTGTAT |
| 3′ UTR- | 33 | VSTFLSRV | 34 | GTTTCCACATTCTTGTCAAGGGTTGGTAGGGTCAGT |
| Ropporin | | GRVSLLNF | | CTTTTAAATTTCTTGCCATTTTAGTGACTGTGCATT |
| | | LPF | | GGTATTTCATTGTGGTTTATTTGCATGATGACTAAT |
| | | | | GCTCAACACCAACTAATCATGTTGAGTATTTTTAAT |
| | | | | GTGCTTATTTGCCACTCATATATCTTCTTTGATGAA |
| | | | | GTGTCTCTTCAAATATTTTGCCCATTTAAAAACTGT |
| | | | | ATTGATTCTTATTATTGAATTGCAATAATTCTTTCT |
| | | | | ATCCGGATATATATCCTTTGCCAGATATGTGTATTA |
| | | | | CAAATGTTTTCTCCTAGCCTTCCACCTCAGCCTCCC |
| | | | | AAGTAGCTGGGAATGCAGGTGTGCACCACCACTCCA |
| | | | | GGGTTTTTTGTTGTTGTTGTTGTTGTTTTTCTGTAG |
| | | | | AGACAGGGTCTTGCCATGCTGCCGAGGCTGCTCTCA |
| | | | | AACTCCTGGGATCAAGAAATCCTCCTGCCTCGGCCT |
| | | | | CCCAAAGTGCTGACATTACAAGCATGAGCCACTGTG |
| | | | | CCTGGCTAACTTTTCATCTTTTAAAGTAGTGTCTTG |
| | | | | CAAAGAACAACATTTTAATGAAGTCCATTTATCAAC |
| | | | | TTTTTGATTCATTGTCCATGCTTTTTGCATAATAAG |
| | | | | AAATCTTTGCCTGCCTCAAAATTGCAAAGCTT |
| Desmocollin 3 | 35 | FRGYLANN | 36 | TTTAGGGGCTATTTAGCAAATAATAAATAAATTGAT |
| | | K | | TTAGAATAGAAGAAATCATGTGTTGGAAAAGAGGCT |
| | | | | TGAAACAAGTTCGGTGTTAGAGAAGAGAATATTAAG |
| | | | | AAACAAGTGGGAGATAGGACTTCTAAATGCTACACT |
| | | | | AAGGATTTCGGATTTATTCTCATCCTAAAGGAGAGC |
| | | | | CAGCCAAGGCTTTTCTACAGGAGAGAGGTATAATCA |
| | | | | AGAAGCGTGAAGCTGAGTCAGTAGGGGGATCAGTGA |
| | | | | GAATAGGAAGACATCAGGGTTGGGGAAGATGAAAGC |
| | | | | TTA |
| BRD2 | 37 | ESRPMSYD | 38 | GAGAGCAGGCCCATGAGTTACGATGAGAAGCGGCAG |
| | | EKRQLSLD | | CTGAGCCTGGACATCAACAAATTACCTGGGGAGAAG |
| | | INKLPGEK | | CTGGGCCGAGTTGTGCATATAATCCAAGCCAGGGAG |
| | | LGRVVHII | | CCCTCTTTACGTGATTCAAACCCAGAAGAGATTGAG |
| | | QAREPSLR | | ATTGATTTTGAAACACTCAAGCCATCCACACTTAGA |
| | | DSNPEEIE | | GAGCTTGAGCGCTATGTCCTTTCCTGCCTACGTAAG |
| | | IDFETLKP | | AAACCCCGGAAGCCCTACAGTACGTATGAAATGAGG |
| | | STLRELER | | TTCATCTCATGGTTCTGAGGACAGTTGAGGAAAGAT |
| | | YVLSCLRK | | GGTGGGGTCTGTTTGCATTCAGGATTGTCAGCTCCC |
| | | KPRKPYST | | AGGATAATGGGATGTGTTGGTTGGCAGCTGACGTTC |
| | | YEMRFISW | | AAGAAGGGAACTTGGGAACCTTAGGGGCCCATAATA |
| | | F | | AGATGCTTGGGGCAATCTTAAGGCTTGCGGCCGCAC |
| | | | | TCGA |
| eIF4G1 | 39 | DPNQGGKD | 40 | GATCCAAACCAAGGAGGAAAGGATATCACAGAGGAG |
| | | ITEEIMSG | | ATCATGTCTGGGGCCCGCACTGCCTCCACACCCACC |
| | | ARTASTPT | | CCTCCCCAGACGGGAGGCGGTCTGGAGCCTCAAGCT |
| | | PPQTGGGL | | AATGGGGAGACGCCCCAGGTTGCTGTCATTGTCCGG |
| | | EPQANGET | | CCAGATGACCGGTCACAGGGAGCAATCATTGCTGAC |
| | | PQVAVIVR | | CGGCCAGGGCTGCCTGGCCCAGAGCATAGCCCTTCA |
| | | PDDRSQGA | | GAATCCCAGCCTTCGTCGCCTTCTCCGACCCCATCA |
| | | IIADPRPGL | | CCATCCCCAGTCTTGGAACCGGGGTCTGAGCCTAAT |
| | | PGPEHSPS | | CTCGCAGTCCTCTCTATTCCTGGGGACACTATGACA |
| | | ESQPSSPS | | ACTATACAAATGTCTGTAGAAGAA |
| | | PTPSPSPV | | |
| | | LEPGSEPN | | |
| | | LAVLSIPG | | |
| | | DTMTTIQM | | |
| | | SVEE | | |

### Cancer Profiling Panels and Methods of Use

Provided herein are cancer profiling panels and methods of their use. Such cancer profiling panels can be used for detecting, pronging, and monitoring cancer, e.g., prostate cancer. The panels as provided herein can be used to analyze one or more antibodies to a plurality of polypeptide probes, such as one or more autoantibodies. A panel allows for the simultaneous analysis of multiple antibodies, such as autoantibodies, to a plurality of polypeptide probes correlating with carcinogenesis and/or metastasis. For example, a panel can include markers identified as correlating with cancerous tissue, metastatic cancer, localized cancer that is likely to metastasize, pre-cancerous tissue that is likely to become cancerous, and pre-cancerous tissue that is not likely to become cancerous. Depending on the subject, panels may be analyzed alone or in combination in order to provide the best possible diagnosis and/or prognosis.

In one embodiment, an antibody profiling panel can comprise a plurality of polypeptide probes, wherein one or more of the probes is capable of binding an antibody. In another embodiment an antibody profiling panel can comprise a plurality of probes, wherein one or more of the probes is capable of binding an antibody that targets a foreign antigen. In another embodiment an antibody profiling panel can comprise a plurality of probes, wherein each of the probes is capable of binding an autoantibody.

In one embodiment, an antibody profiling panel comprises at least 2-100 probes, 50-200 probes, 100-500 probes, 200-750 probes, 200-1000 probes, 2-5,000 probes or 2-10,000 probes. In one embodiment, an antibody profiling panel comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 polypeptide probes. In another embodiment, an antibody profiling panel comprises at least about 50, 100, 150, 200, 250, 500, 750, 1000, 5000, 10,000, 15,000, 20,000, 25,000, 30,000, 40,000, 50,000, 60,000, 70,000, 75,000, or 100,000 polypeptide probes. In one embodiment, the probes are polypeptide probes. In another embodiment, the probes are molecules that mimic an epitope bound by a particular antibody. In one embodiment, the probes are an epitope fragment of the polypeptide.

Disclosed herein are cancer profiling panels comprising a plurality of polypeptide probes, at least one of which is a polypeptide probe comprising a full length or epitope fragment of a protein comprising an amino acid sequence, or encoded by a nucleotide sequence, disclosed in Table 1. Such cancer profiling panels may comprise 1, 2, 3, 4, 5, 6, 7, 8, 9, 10, 11, 12, 13, 14, 15, 16, 17, 18, 19, or 20 polypeptide probes comprising a full length or epitope fragment of a protein comprising an amino acid sequence, or encoded by a nucleotide sequence, disclosed in Table 1.

Also disclosed herein are cancer profiling panels comprising a plurality of polypeptide probes, at least one of which is a polypeptide probe comprising a full length or epitope fragment of a protein comprising an amino acid sequence, or encoded by a nucleotide sequence, having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or 100% identity with a sequence disclosed in Table 1.

### Methods of Measuring Antibodies

Disclosed herein are methods comprising contacting a sample from a subject with a cancer profiling panel comprising a plurality of polypeptide probes, at least one of which is a polypeptide probe comprising a full length or epitope fragment of a protein comprising an amino acid sequence, or encoded by a nucleotide sequence, disclosed in Table 1, and measuring a level of antibodies from the sample bound to the plurality of polypeptide probes. Such cancer profiling panels may comprise 1, 2, 3, 4, 5, 6, 7, 8, 9, 10, 11, 12, 13, 14, 15, 16, 17, 18, 19, or 20 polypeptide probes comprising a full length or epitope fragment of a protein comprising an amino acid sequence, or encoded by a nucleotide sequence, disclosed in Table 1.

Also disclosed herein are methods comprising contacting a sample from a subject with a cancer profiling panel comprising a plurality of polypeptide probes, at least one of which is a polypeptide probe comprising a full length or epitope fragment of a protein comprising an amino acid sequence, or encoded by a nucleotide sequence, having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or 100% identity with a sequence disclosed in Table 1, and measuring a level of antibodies from the sample bound to the plurality of polypeptide probes.

The level, presence or absence of an antibody can be determined by detecting the binding of one or more autoantibodies to a polypeptide probe. Detection of an antibody can be either quantitative or qualitative. For quantitative assays, the amount of antibody detected can be compared to a control or reference to determine whether an antibody is overexpressed or underexpressed in a sample. For example, the control or reference can be a normal sample or a sample from a known disease state, such as a cancer sample.

Antibody binding to a polypeptide probe can be detected by techniques known in the art, such as, but not limited to, radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays. Any of the assays used can be quantitative or qualitative, as desired.

Detection of an antibody bound to a polypeptide probe can be detected using labeling technology. For example, one or more antibodies in a sample collected from a subject to be tested can be directly labeled (e.g., with a fluorescent or radioactive label) and exposed to a polypeptide probe or probe panel. Detection of a signal from the interaction can be achieved using methodology appropriate to the type of label used (e.g., fluorescent microscopy can be used to detect binding of a fluorescently labeled autoantibody to a polypeptide probe). In one embodiment, an autoantibody is detected by detecting binding of a labeled secondary antibody or other antibody-binding reagent which specifically binds to the antibody bound to the polypeptide probe (e.g., a “sandwich immunoassay”). Many methods are known in the art for detecting binding in an immunoassay and are within the scope of the present disclosure.

In one embodiment, the immunoassay described in U.S. Pat. Nos. 5,599,677, 5,672,480, or both, each of which is herein incorporated by reference, is used. In one embodiment, automation is utilized to detect binding of one or more autoantibodies to a polypeptide probe or probe panels. Methods for the automation of immunoassays include those described in U.S. Pat. Nos. 5,885,530, 4,981,785, 6,159,750, and 5,358,691, each of which is herein incorporated by reference.

Analysis and/or presentation of results can also be automated. In one embodiment, a computer with software that analyzes raw data and generates a prognosis, diagnosis, or active monitoring based on the level, presence or absence of antibody binding to one or more polypeptide probes is used. A computer-based analysis program can be used to translate the raw data generated by the detection assay (e.g., a presence, absence, or amount of antibody binding to one or more polypeptide probes) into data of predictive value for a clinician. The clinician can access the predictive data using any suitable means. In one embodiment, the data is transmitted over a network. In another embodiment, the data is accessible by a clinician.

Any method capable of receiving, processing, and transmitting the information to and from a laboratory conducting the assay, medical personnel, and a subject can be used. In one embodiment, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. In one embodiment, the sample comprises a tissue or other biological sample and the subject visits a medical center to have the sample obtained and sent to the profiling center. In another embodiment, a subject collects the sample themselves (e.g., a buccal swab) and directly sends it to a profiling center. In another embodiment, the sample comprises previously determined biological information. The information can be directly sent to the profiling service by the subject (e.g., an information card containing the information may be scanned by a computer and the data transmitted to a computer of the profiling center using an electronic communication system). Upon being received by the profiling service, a sample can be processed and a profile produced (i.e., antibody level, presence or absence of antibody). A profile generated can be specific for the diagnostic, prognostic, or active monitoring information desired for a subject.

Profile data can be prepared in a format suitable for interpretation by a treating clinician. In one embodiment, rather than providing raw expression data, the prepared format represents a diagnosis, screening or risk assessment (e.g., likelihood of metastasis or PSA failure or the development of high prostate specific antigen levels in a patient following prostate cancer therapy (e.g., surgery)) for the subject, along with recommendations for particular treatment options. The data can be displayed to the clinician by any suitable method. In one embodiment, the profiling service generates a report that is printed for the clinician (e.g., at the point of care). In another embodiment, the report is displayed to the clinician on a computer monitor.

In one embodiment, the information is first analyzed at the point of care or at a regional facility. The raw data is then sent to a central processing facility for further analysis. In one embodiment, further analysis comprises converting the raw data to information useful for a clinician or subject, such as a patient. The central processing facility can provide the advantage of privacy (all data is stored in a central facility with uniform security protocols), speed, and uniformity of data analysis. The central processing facility can also control the fate of the data following treatment of a subject. In one embodiment, using an electronic communication system, the central facility provides data to the clinician, the subject, researchers, or any other individual. In one embodiment, a subject is able to directly access the data using the electronic communication system. In another embodiment, a subject chooses further intervention or counseling based on the result. In one embodiment, the data is used for research use. The data can be used to further optimize the inclusion or elimination of markers as useful indicators of a particular condition or stage of disease.

Due to the low specificity of the prostate-specific antigen (PSA) assay and a high false positive rate, a large number of prostate cancer (PCA) biopsies may be performed unnecessarily. Consequently, there is a need for new biomarkers that can identify PCA at any stage of progression while limiting the number of false positives. The present disclosure provides an effective method to solve this problem by use of autoantibody signature-developed biomarkers. Autoantibody signature refers to a molecular “fingerprint” of autoantibodies produced against a disease state.

A presence of an immune response to a specific protein expressed in cancerous cells can be indicative of a presence of cancer. Accordingly, the present disclosure provides a method (e.g., diagnostic or screening method) for detecting a presence of an antibody, such as an autoantibody, to a tumor or tumor-associated antigen. In one embodiment, the presence of an antibody in cancerous but not cancerous cells is indicative of the presence of cancer. In one embodiment, the antibody is an antibody to a tumor antigen.

A method or composition disclosed herein can find utility in the diagnosis, screening, or characterization of a cancer. In one embodiment, a presence of an antibody, such as an autoantibody, to a specific protein can be indicative of a cancer. In another embodiment, detection of an antibody in a sample, such as an autoantibody, can be indicative of a specific stage or sub-type of the same cancer. The information obtained by detecting an antibody as described herein can be used to determine a prognosis, diagnosis or active monitoring, wherein an appropriate course of treatment can be determined. In another embodiment, a subject with a specific antibody or stage of cancer can respond differently to a given treatment than individuals lacking the antibody. The information obtained from a method disclosed herein can thus provide for the personalization of diagnosis and treatment.

### Panel/Kit

The present disclosure further provides panels and kits for the detection of markers. In some embodiments, the presence of a cancer marker is used to provide a prognosis to a subject. The information provided is also used to direct the course of treatment. For example, if a subject is found to have a marker indicative of a highly metastasizing tumor, additional therapies (e.g., hormonal or radiation therapies) can be started at a earlier point when they are more likely to be effective (e.g., before metastasis). In addition, if a subject is found to have a tumor that is not responsive to hormonal therapy, the expense and inconvenience of such therapies can be avoided.

In some embodiments, the present invention provides a panel for the analysis of a plurality of markers. The panel allows for the simultaneous analysis of multiple markers correlating with carcinogenesis and/or metastasis. For example, a panel may include markers identified as correlating with cancerous tissue, metastatic cancer, localized cancer that is likely to metastasize, pre-cancerous tissue that is likely to become cancerous, and pre-cancerous tissue that is not likely to become cancerous. Depending on the subject, panels may be analyzed alone or in combination in order to provide the best possible diagnosis and prognosis. Markers for inclusion on a panel are selected by screening for their predictive value using any suitable method, including but not limited to, those described in the illustrative examples below.

### EXAMPLES

These examples are provided for illustrative purposes only and not to limit the scope of the claims provided herein.

### Example 1. Discovery of Biomarkers for Prostate Cancer Detection

The patient cohorts (obtained from the University of Michigan, Johns Hopkins University, and Bioreclamation) were composed of two arms: 414 men aged 40 to 70 with PSA between 2.5 and 20 ng/ml diagnosed with PCA and 346 men aged 25 to 40 with PSA<1.0 ng/ml who were self-reported to be cancer-free and had no familial PCA history. Young men were selected as the negative cohort based on concerns that men aged 40 to 70 could potentially have undiagnosed PCA without elevated PSA. These cohorts were selected since it has been documented that 40% of men aged 40 to 50 can have undiagnosed, asymptomatic PCA. The incidence increases by 10% for each subsequent decade. The two arms of the study were therefore composed of biopsy-positive males (diseased) and low-PSA, non-PCA family or personal history men under the age of 40 (clinically healthy). Because older men can have undiagnosed PCA, the lower aged men were used to decrease the possibility of having samples that were equivocal for PCA and to increase the accuracy and utility of the biomarkers selected and the algorithm developed.

Phage libraries that had been developed by iterative biopanning were used for further screening. A total of 62 biomarkers were evaluated for relative signal compared to background, and the marker number was reduced to 18 where the strongest signal-to-noise ratio over multiple samples was seen. The 18 biomarkers were attached to Luminex MagPlex beads and tested against training (N=519, PCA=268, Healthy=251) and validation (N=259, PCA=146, Healthy=113) cohorts. Retrospective samples for the secohorts were obtained from three different sources (the University of Michigan, Johns Hopkins University, and Bioreclamation). The analyte tested, immunoglobulin, is very robust and not prone to degradation with a simple freeze-thaw cycle. Carboxyl chemistry was used to bind the phage to the beads, according to the product protocol (Luminex). Samples were tested in triplicate with high and low serum controls included on each plate and high and low calibrator beads included in every sample. The triplicates were averaged to obtain a single value for each sample. The training cohort was tested with 18 biomarkers to discern healthy from diseased patients.

Logistic regression analysis determined the optimal panel to be a total of eight biomarkers. The eight biomarker panel was then used on a separate and distinct validation patient cohort to assess performance characteristics of the diagnostic algorithm.

Samples were randomly divided into a training set (⅔, N=519) and a validation set (⅓, N=259). These two sets were checked for comparability with respect to age, race, and the date each sample was run. Raw biomarker values were normalized by taking the ratio or absolute difference from T7 or BSA. Logistic regression models were used to model the probability of a sample being cancer as a function of the potential biomarkers. Receiver Operating Characteristic (ROC) curves, based on fitted multivariate logistic regression models, were also generated to show the relation between sensitivity and specificity for the range of possible cut points.

To determine the optimal number of biomarkers to include in the final model, following process was used. For each number of biomarkers N=1 to 18 and each normalization method (a total of five including unnormalized), the best fitting model with N biomarkers was calculated along with an estimate of the AUC from leave-one-out cross-validation. No interactions were considered. Although the cross-validated estimates of AUC (area under the ROC curve) were similar for models with six to nine biomarkers, the numerically highest cross-validated estimate of AUC was obtained with eight biomarkers using the T7 difference normalization method. Thus, the final model selected for validation was obtained as the best fitting model (on the training data) containing eight biomarkers using the T7 difference normalization.

A biomarker signature was defined as the linear combination of the eight selected biomarkers each multiplied by their parameter coefficient from the fitted logistic regression model. The value of this signature was calculated for patients in the validation set, and a logistic regression model was used to test its statistical significance. Sensitivity and specificity associated with this signature, when applied to the validation set, were calculated at various possible cut points. The nonparametric estimate of the AUC was also calculated from the validation set. Prevalence-adjusted PPV and NPV were calculated at various possible positivity thresholds using the estimated sensitivity and specificity and an assumed prevalence of cancer equal to 18%. All analyses were performed using SAS V9.3 (Cary, N.C.).

### Example 2. Improved Identification of Prostate Cancer

The patient cohorts (obtained from the University of Michigan, Johns Hopkins University, and Bioreclamation) were composed of two arms: 414 men aged 40 to 70 with PSA between 2.5 and 20 ng/ml diagnosed with PCA and 346 men aged 25 to 40 with PSA<1.0 ng/mL who were self-reported to be cancer-free and had no familial history.

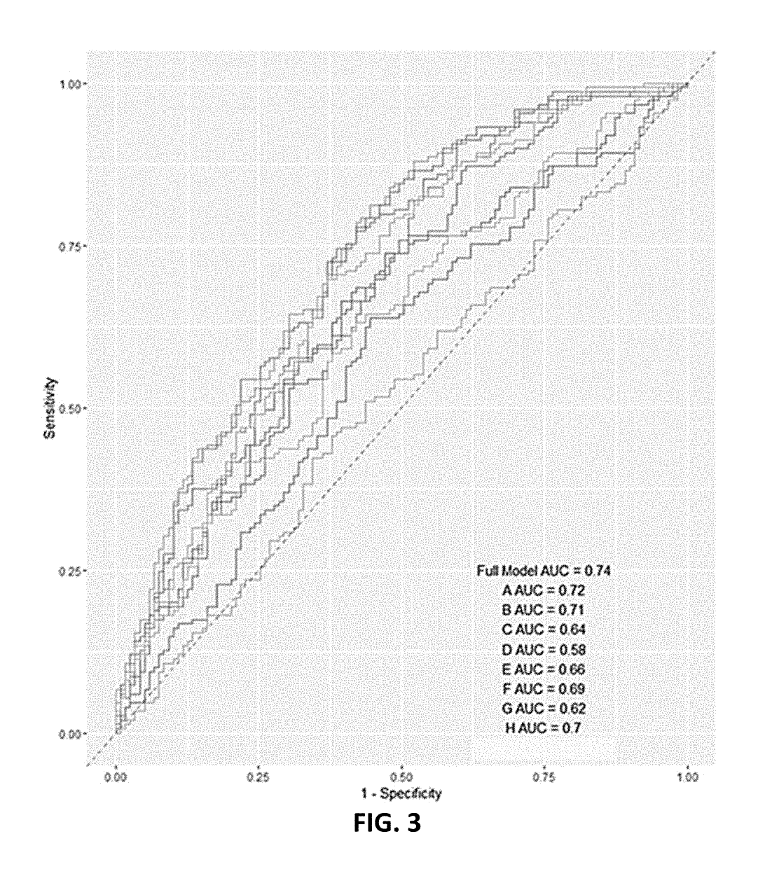

The autoantibody assay was compared with biopsy outcomes in 268 patients at risk of prostate cancer and undergoing prostate biopsy from two academic sites and one US community clinical site. Eligible men were prostate cancer free, 40 years or older, undergoing a prostate biopsy due to a suspicious digital rectal examination finding and/or elevated PSA. The predictive ability of the autoantibody assay was evaluated using the area under AUC to predict PCA vs. no cancer on biopsy and high-grade PCA defined as GS7 or greater from GS6 and no cancer on biopsy. Analyses were repeated restricting to those with PSA 2-10 ng/mL and stratifying by age (<65 vs. ≥65). The autoantibody assay was compared with biopsy outcome in 75 patients with PSA levels of 0.1 to 4 ng/mL. The predictive ability of the assay using the AUC was evaluated in discrimination of GS6 and high-grade cancers from patients who were negative on initial biopsy. FIG. 3 shows ROC curve for case prediction for normalized biomarkers. The estimated area under the ROC curve (AUC) with 95% confidence interval was 0.74, and standard of care (SOC) AUC was 0.535. Utilizing a prevalence rate of 18%, the current cut-point analysis showed that sensitivity was 92.6% and specificity was 35.3%. Negative predictive value (NPV) was 95.4% and positive predictive value (PPV) was 24%.

Analysis of men with PSA between 2-10 ng/mL (N=223) showed an AUC of 0.73 using the antibody assay. The SOC was 0.53. Analysis based on age was performed in this subgroup. In men younger than 65 years old (N=174), the AUC of the biomarkers was 0.75, and SOC was 0.52. In men 65 years or older (N=94), the AUC of the biomarkers was 0.75, and SOC was 0.65. Therefore, the antibody assay as described herein outperformed SOC in both older and younger men and within the diagnostic grey zone of PSA 2-10 ng/mL.

In men with PSA less than 4 ng/mL (N=75; 38 patients with cancer and 37 biopsy negative patients) in the initial target population with PSA between 0.1 and 4.0 ng/mL (median age 61.43 years and median PSA level 3.2 ng/mL and biopsy), the autoantibody assay plus SOC showed discrimination between GS6 or greater from patients negative on initial biopsy (AUC 0.733, 95% CI, 0.69-0.80). SOC AUC was 0.51 (95% CI, 0.49-0.53, p<0.0001). The serum based autoantibody away as described herein was clearly associated with improved identification of patients with higher-grade prostate cancer among men with PSA levels less than 4 ng/mL.

### Example 3. Novel Autoantibody Signature to Predict Risk of High-Grade Prostate Cancer

### Validation Study

Retrospective serum samples and annotated clinical information for 296 biopsy confirmed patients were sourced from two academic sites and one community facility in the United States. Eligible participants included men aged 40 years or older, undergoing a prostate biopsy due to suspicious digital rectal examination finding and/or elevated PSA level. Only men with previously diagnosed prostate cancer were excluded from the study. The men had an average age of 63.3 and an average PSA of 6.0 ng/mL. Twenty-nine percent of men had a PSA<4.0 ng/mL, 60% had a PSA between 4 to 10 ng/mL, and only 10% of men had a PSA greater than 10 ng/mL. The majority (61%) of the men were Caucasian, 6% were African American and in 32% of samples, no race was specified; they were therefore classified as ‘other’. Twenty-seven percent of men had Gleason 6 cancer, 29% had Gleason 7 (3+4) cancer, 7% had Gleason 7 (4+3) cancer, and 20% of men had Gleason 7 cancer with no breakout. Finally, 17% of men had Gleason 8 or greater. Almost all men (98%) in the biopsy positive group had only one biopsy. Put another way, only 2% or a total of three men, underwent repeat biopsy. In the control group, 70% of men had one biopsy, 12% had two biopsies, 8% had three biopsies, and 10% of men had four or more biopsies.

Retrospective samples for these cohorts were obtained from three different sources (the University of Michigan, Johns Hopkins University, and the Michigan Institute of Urology). All samples in the autoantibody-binding peptides discovery study, the training set and the validation study were prostate cancer positive and biopsy negative controls and all cohorts were age and PSA matched. Samples were shipped to a central laboratory and processed or stored upon receipt. No patients were compensated for participating. Test results were not provided to the clinical sites for patient care, and the laboratory technicians who performed the biomarker tests were blinded for patient characteristics. The autoantibody-binding peptides discovery study, the training set study and the validation study were all performed in accordance with the Standards for Reporting of Diagnostic Accuracy criteria.

### Novel Serum Based Multiplexed Autoantibody Assay Development

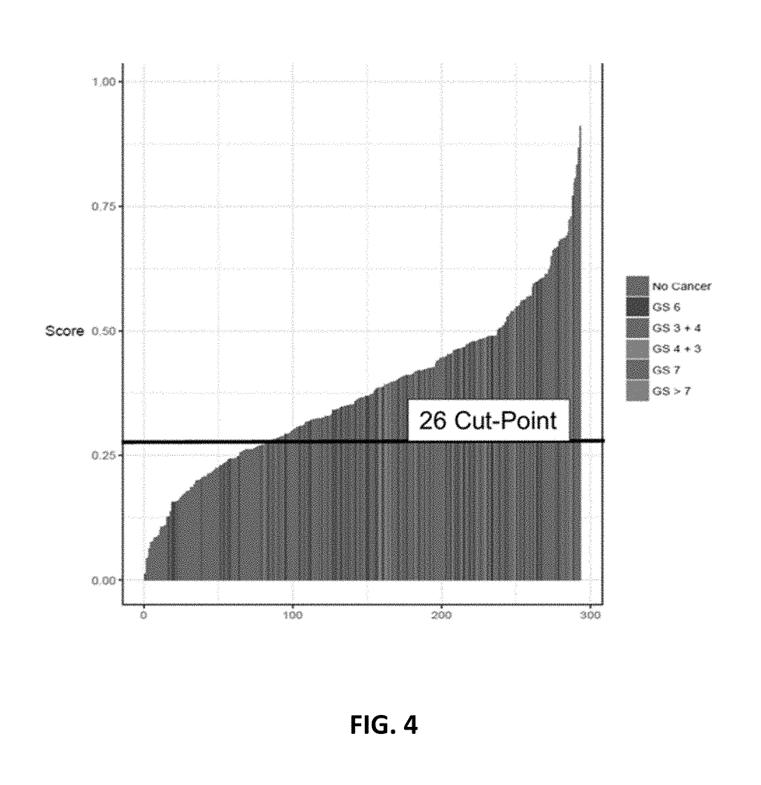

State-of-the-art genomic and proteomic information was utilized to develop and refine an assay that can quantify the presence and aggressiveness of prostate cancer (FIG. 4). A total of 268 samples in the autoantibody-binding peptide discovery and assessment study were collected from prostate cancer positive and biopsy negative controls. Twenty-one phage-peptides were analyzed to determine the specific biological markers known to be associated with an immune system response to aggressive prostate cancer.

Using a training set of 196 subjects, a unique algorithm was developed to discern between men with low- and high-grade cancers independent of PSA levels. The final algorithm incorporated the ten autoantibody binding peptides prioritized for aggressive cancer detection: ARF6, NKX3-1, 5′-UTR-BMI1, 3′-UTR-Ropporin, AURKAIP-1, CSNK2A2, hnRNAPA1, KDELR1, SMAD3, BRD2 and FKBP4. The assay and the classifier algorithm using the 10 specific autoantibody-binding peptides optimizes the negative predictive value and minimizes false negative results for Gleason 7+ disease. Finally, a validation analysis using independent samples from 296 subjects assessed the performance of the diagnostic algorithm.

The primary objective was to validate the accuracy of the serum-based multiplexed autoantibody assay to predict high-grade prostate cancer for men aged 40 years or older. Results of the studies were analyzed by independent biostatisticians. Because the immune system continuously changes as cancer risk fluctuates, the performance of the peptides was assessed and evaluated as area under the curve (AUC) of the receiver operating characteristic (ROC) curve. The 95% confidence intervals (CIs) and comparisons of AUCs were determined using DeLong's method as implemented in the R package pROC. The combination and predictive value of the multiplexed autoantibody assay was modeled by logistic regression analysis. The assay aimed to identify high-grade (Gleason Score ≥7) prostate cancer. The control group included men who either did not have cancer or men who had low-grade cancer (Gleason Score ≤6), wherein active surveillance (i.e., no treatment) is the preferred management strategy. The logistic regression model was then applied to estimate the probability of high-grade prostate cancer on biopsy.

Using T7 phage with epitope inserts, autoantibodies against peptides/inserts derived from prostate cancer were identified. At the completion of the biomarker discovery study, samples were randomly divided into a training set (N=196) and a validation set (N=296). These two sets were checked for comparability with respect to age, race, and the date each sample was run. Raw biomarker values were normalized against an internal standard control sample. Logistic regression models were used to model the probability of a sample being high grade cancer as a function of the potential biomarkers. ROC curves based on fitted multivariate logistic regression models, were also generated to show the relation between sensitivity and specificity for the range of possible cut points.

To determine the optimal number of peptides to include in the final model, following process was used. For each number of peptides N=1 to 21 and each normalization method (a total of two including unnormalized) the model with the highest discrimination (based on AUC) with N peptides was calculated. No interactions were considered. Although the cross-validated estimates of AUC were similar for a model with twelve peptides, the numerically highest cross-validated estimate of AUC was obtained with ten peptides using the T7 ratio normalization method. To normalize the peptides with T7, for each subject we divided each peptide by that subject's T7 value. Thus, the final model selected for validation was obtained as the best discriminating model (on the training data) containing ten peptides using the T7 ratio normalization.

An estimate of predicted risk was defined as the linear combination of the ten selected peptides each multiplied by their parameter coefficient from the fitted logistic regression model. This risk was calculated for patients in the validation set, and a table of model performance statistics was generated using each risk estimate in the validation data set as a decision threshold for the prediction of high-grade cancer. Specifically, sensitivity, specificity, positive predictive value (PPV) and negative predictive value (NPV) were calculated at all present cut points. In addition, PPV and NPV were calculated using test sensitivity and specificity at the US average incidence of prostate cancer defined prevalence of 18%. All analysis was performed using R 3.32 (R Core Team, Vienna, Austria).

### Example 4. Antibody Signature for Prostate Cancer Prognosis Panel

A cancer profiling panel and related test is developed for risk assessment for aggressive disease in patients with a PSA 2-10 ng/mL and considering an initial biopsy or follow-up to a negative biopsy. The cancer profiling panel and related test differentiates between Gleason 6 and 7 or higher cancers among all patients, thus allowing a clinician to perform an initial/follow-up biopsy or routine monitoring without a biopsy.

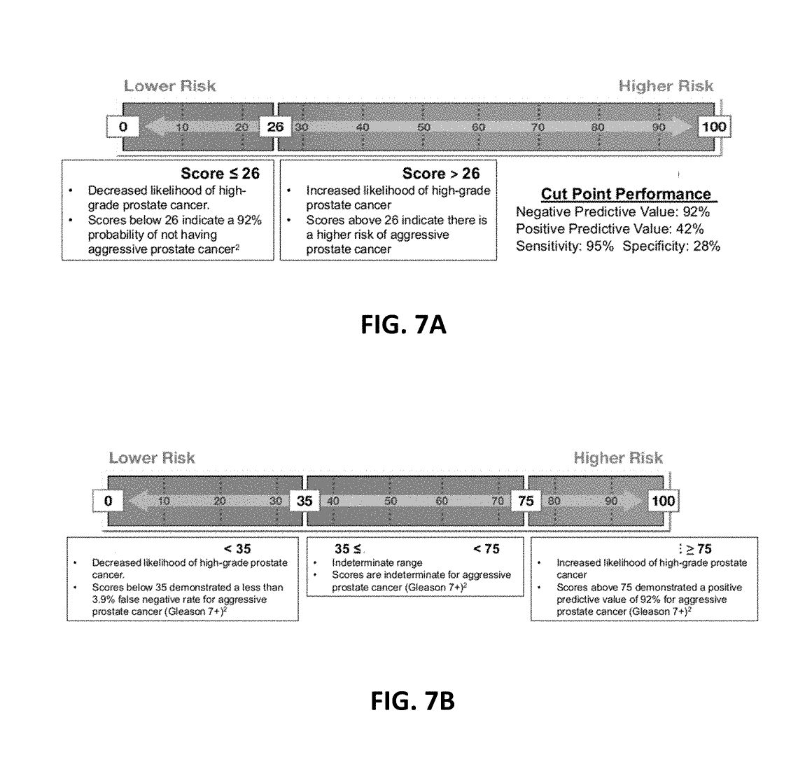

A risk assessment score is presented on a scale from 0 to 100 with a single cut point at 26 (FIG. 7A) and calculated using an algorithm based on a measurement of the level of antibodies bound to a cancer profiling panel comprising a plurality of polypeptide probes; for example, the probes in Table 2. A score less than or equal to 26 shows decreased likelihood of high-grade prostate cancer, e.g., a 92% probability of not having aggressive prostate cancer. A score greater than 26 shows increased likelihood of high-grade prostate cancer.

Model coefficients for 10 markers are listed in Table 2. The names of the markers correspond to marker names in Table 1. It is reasonable to interpret the coefficients as weights. The risk estimate is a weighted sum of biomarker concentrations.

TABLE 2
Model coefficients of the cancer prognosis biomarkers

| Marker | Model Coefficients |
|---|---|
| NKX3-1 | 2.782082 |
| ARF6 | −1.623837 |
| AURKAIP1 | −0.491664 |
| CSNK2A2 | −0.619283 |
| UTR-BMI1 | −3.649535 |
| 3′ UTR-Ropporin | −1.198423 |
| hnRNPA1 | 0.262977 |
| KDELR1 | 2.595930 |
| SMAD3 | −0.170078 |
| BRD2 | 2.938935 |

### Example 5. Antibody Signature for Prostate Cancer Active Surveillance Panel

A cancer profiling panel and related test is developed for active monitoring and risk assessment for men with non-aggressive disease who are not receiving treatment. The cancer profiling panel and related test differentiates between Gleason 6 and 7 or higher cancers among patients who had positive biopsy.

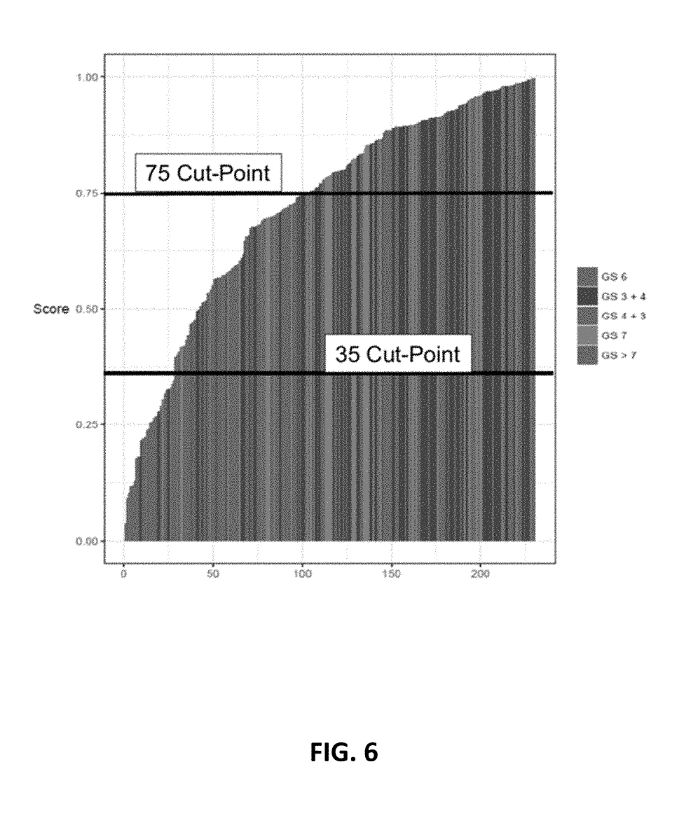

A risk assessment score is presented on a scale from 0 to 100 with a higher risk cut point at 75 and a lower risk cut point at 35 (FIG. 6). The score is calculated using an algorithm based on a measurement of the level of antibodies bound to a cancer profiling panel comprising a plurality of polypeptide probes; for example, the probes in Table 3. A score less than 35 shows decreased likelihood of high-grade prostate cancer, for example, a less than 3.9% false positive rate for aggressive prostate cancer (Gleason 7+) (FIG. 7B). A score greater than or equal to 75 shows an increased likelihood of high-grade prostate cancer; for example, a score above 75 can demonstrate a positive predictive value of 92% for aggressive prostate cancer.

The model coefficients for 12 markers are listed in Table 3. It is reasonable to interpret the coefficients as weights. The risk estimate is a weighted sum of biomarker concentrations.

TABLE 3
Model coefficients of the cancer active surveillance biomarkers

| Marker | Model Coefficients |
|---|---|
| NKX3-1 | −0.75422 |
| ARF6 | −1.85224 |
| AURKAIP1 | 1.72488 |
| CSNK2A2 | −0.84910 |
| UTR-BMI1 | −6.09839 |
| CEP164 | 3.56158 |
| 3′ UTR-Ropporin | −4.22140 |
| hnRNPA1 | 1.29073 |
| KDELR1 | 5.59891 |
| FKBP4 | 2.59133 |
| SMAD3 | −1.04672 |
| BRD2 | −0.00718 |

1. A cancer profiling panel comprising a plurality of polypeptide probes, at least one of which comprises:
(a) a polypeptide probe comprising the full length or epitope fragment of a protein comprising amino acid sequence SEQ ID NO: 23 or encoded by SEQ ID NO: 24;
(b) a polypeptide probe comprising the full length or epitope fragment of a protein comprising amino acid sequence SEQ ID NO: 25 or encoded by SEQ ID NO: 26;
or
(c) a polypeptide probe comprising the full length or epitope fragment of a protein comprising amino acid sequence SEQ ID NO: 27 or encoded by SEQ ID NO: 28.

2. (canceled)

3. The cancer profiling panel of claim 1, comprising two or more polypeptide probes selected from the group consisting of: (a), (b), and (c).

4. The cancer profiling panel of claim 1, comprising polypeptide probes (a), (b), and (c).

5-15. (canceled)

16. The cancer profiling panel of claim 1, wherein the polypeptide probes are, individually, displayed on a phage.

17. The cancer profiling panel of claim 16, wherein the phage is a T7 phage.

18. The cancer profiling panel claim 1, wherein the polypeptide probes are configured to be specifically bound by an antibody.

19. The cancer profiling panel of claim 18, wherein the antibody is a human antibody.

20. The cancer profiling panel of claim 19, wherein the antibody is an autoantibody.

21. The cancer profiling panel of claim 1, wherein the polypeptide probes are arranged in an addressable array.

22. (canceled)

23. The cancer profiling panel of claim 1, wherein the polypeptide probes are attached to a bead.

24. The cancer profiling panel of claim 1, wherein the polypeptide probes are attached to a man-made substrate.

25-30. (canceled)

31. A method comprising contacting a sample from a subject with a cancer profiling panel comprising a plurality of polypeptide probes, at least one of which comprises:
(a) a polypeptide probe comprising the full length or epitope fragment of a protein comprising amino acid sequence SEQ ID NO: 23 or encoded by SEQ ID NO: 24;
(b) a polypeptide probe comprising the full length or epitope fragment of a protein comprising amino acid sequence SEQ ID NO: 25 or encoded by SEQ ID NO: 26;
or
(c) a polypeptide probe comprising the full length or epitope fragment of a protein comprising amino acid sequence SEQ ID NO: 27 or encoded by SEQ ID NO: 28;
and
measuring a level of antibodies from the sample bound to the plurality of polypeptide probes.

32. (canceled)

33. The method of claim 31, wherein the cancer profiling panel comprises two or more polypeptide probes selected from the group consisting of: (a), (b), and (c).

34. The method of claim 31, wherein the cancer profiling panel comprises polypeptide probes (a), (b), and (c).

35-45. (canceled)

46. The method of claim 31, wherein the polypeptide probes are, individually, displayed on a phage.

47-48. (canceled)

49. The method of claim 31, wherein the antibodies comprise human antibodies.

50. The method of claim 49, wherein the human antibodies comprise autoantibodies.

51. The method of claim 31, wherein the polypeptide probes are arranged in an addressable array.

52. (canceled)

53. The method of claim 31, wherein the polypeptide probes are attached to a bead.

54-88. (canceled)

89. The method of claim 31, wherein the sample is a blood or serum sample.
